(19) United States
(12) Patent Application Publication
(10) Pub. No.: US 2026/0170119 A1
(43) Pub. Date: Jun. 18, 2026

(54) AEROSOL-GENERATING DEVICES UNLOCKABLE WITH WIRELESS COMMUNICATIONS

(71) Applicant: Altria Client Services LLC, Richmond, VA (US)

(72) Inventors: Andre SOTO, Plano, TX (US); Rangaraj SUNDAR, Midlothian, VA (US); Nedko NEDEV, Stevenage (GB); Jack HODGES, Biggleswade (GB); Guillaume MICHEL, Cambridge (GB); Niall GALLAGHER, Cambridge (GB)

(73) Assignee: Altria Client Services LLC, Richmond, VA (US)

(21) Appl. No.: 19/417,618

(22) Filed: Dec. 12, 2025

Related U.S. Application Data
(60) Provisional application No. 63/733,706, filed on Dec. 13, 2024.

(51) Int. Cl. G06F 21/35; A24F 40/46; A24F 40/53; A24F 40/65; A61M 15/00; A61M 15/06; G06F 21/32; G06Q 20/34; G06Q 20/40

(52) U.S. Cl. CPC G06F 21/35; A24F 40/46; A24F 40/53; A24F 40/65; A61M 15/009; G06F 21/32; G06Q 20/352; G06Q 20/40145; A61M 15/06

(57) ABSTRACT

A system for unlocking an aerosol-generating device, includes a physical document issued and unique to an adult consumer. The physical document includes at least one first wireless communication chip and memory configured to store verified personal data of the adult consumer. The system further includes an aerosol-generating device having at least one second wireless communication chip and a controller. The controller is configured to receive, from the physical document via the first wireless communication chip and the second wireless communication chip, the verified personal data of the adult consumer, determine whether the adult consumer meets an age condition based on the verified personal data, and unlock the aerosol-generating device in response to the adult consumer meeting the age condition.

### CROSS REFERENCE TO RELATED APPLICATIONS

This application claims the benefit of U.S. Provisional Application No. 63/733,706 filed on Dec. 13, 2024. The entire disclosure of the above application is incorporated herein by reference.

### BACKGROUND

### Field

The present disclosure relates to aerosol-generating device unlockable with wireless communications.

### Description of Related Art

Aerosol-generating devices generally heat a material to a temperature sufficient to release constituents of the material. Such aerosol-generating devices may include electronic-vaping devices that heat a pre-vapor formulation and heated tobacco devices that heat an aerosol-forming substrate. Some aerosol-generating devices may be in a locked state to prevent unauthorized consumers from using the devices. Such devices may be unlocked via wireless communications, such as Bluetooth® communication, near-field communications (NFC), Wi-Fi communication, and radio frequency identification (RFID) communications.

### SUMMARY

At least some example embodiments relate to systems for unlocking aerosol-generating devices.

In some example embodiments, a system for unlocking an aerosol-generating device, includes a physical document issued and unique to an adult consumer. The physical document includes at least one first wireless communication chip and memory configured to store verified personal data of the adult consumer. The system further includes an aerosol-generating device including at least one second wireless communication chip and a controller. The controller is configured to receive, from the physical document via the first wireless communication chip and the second wireless communication chip, the verified personal data of the adult consumer, determine whether the adult consumer meets an age condition based on the verified personal data, and unlock the aerosol-generating device in response to the adult consumer meeting the age condition.

In some example embodiments, a system for unlocking an aerosol-generating device, includes an operator terminal having a digital wallet application configured to store a mobile identification containing verified personal data unique to an adult consumer, and an aerosol-generating device having a controller. The controller is configured to receive, from the operator terminal via a wireless communication, the verified personal data in the mobile identification, determine whether the adult consumer meets an age condition based on the verified personal data, and unlock the aerosol-generating device in response to the adult consumer meeting the age condition.

At least some example embodiments relate to methods for unlocking aerosol-generating devices.

In some example embodiments, a method for unlocking an aerosol-generating device, includes receiving, from a wireless communication chip of a physical document issued and unique to an adult consumer, verified personal data of the adult consumer, determining whether the adult consumer meets an age condition based on the verified personal data, and unlocking the aerosol-generating device in response to the adult consumer meeting the age condition.

In some example embodiments, a method for unlocking an aerosol-generating device with a mobile identification unique to an adult consumer and stored on an operator terminal, includes receiving, via a wireless communication, verified personal data in the mobile identification unique to the adult consumer and stored in a digital wallet application of the operator terminal, determining whether the adult consumer meets an age condition based on the verified personal data, and unlocking the aerosol-generating device in response to the adult consumer meeting the age condition.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a block diagram of an example system including an aerosol-generating device and an operator terminal for unlocking the aerosol-generating device, in accordance with at least one example embodiment.

FIG. 2 is a diagram of an example interaction between the operator terminal and the aerosol-generating device of FIG. 1 when the aerosol-generating device is in a locked state, according to one or more example embodiments.

FIG. 3 is a front view of the operator terminal of FIG. 1 including an interactive user interface, according to one or more example embodiments.

FIG. 4 is a front view of the operator terminal of FIG. 1 including a pass storable in a digital wallet application, according to one or more example embodiments.

FIG. 5 is a diagram of an example interaction between the operator terminal and the aerosol-generating device of FIG. 1 for unlocking the aerosol-generating device, according to one or more example embodiments.

FIG. 6 is a block diagram of another example system including an aerosol-generating device and an operator terminal for unlocking the aerosol-generating device, in accordance with at least one example embodiment.

FIG. 7 is a diagram of the operator terminal of FIG. 6 including an interactive user interface, according to one or more example embodiments.

FIG. 8 is a block diagram of another example system including an aerosol-generating device and a consumer payment card for unlocking the aerosol-generating device, in accordance with at least one example embodiment.

FIG. 9 is a block diagram of another example system including an aerosol-generating device and a consumer biometric identification for unlocking the aerosol-generating device, in accordance with at least one example embodiment.

FIG. 10 is a block diagram of another example system including multiple aerosol-generating devices and an operator terminal, according to one or more example embodiments.

FIG. 11 is a block diagram of another example system including an aerosol-generating device and multiple operator terminals, according to one or more example embodiments.

FIG. 12 is a side view of an example electronic-vaping device employable as any one of the aerosol-generating devices herein, according to one or more example embodiments.

FIG. 13 is a side view of an example heated tobacco device employable as any one of the aerosol-generating devices herein, according to one or more example embodiments.

FIG. 14 is a side view of an example consumable receiving aerosol-generating device employable as any one of the aerosol-generating devices herein, according to one or more example embodiments.

FIGS. 15-16A-B illustrate control methods for an operator terminal to unlock an aerosol-generating device, according to one or more example embodiments.

FIGS. 17-19 illustrate control methods for unlocking an aerosol-generating device, according to one or more example embodiments.

FIG. 20 illustrates a control method for verifying a consumer and generating a pass for unlocking an aerosol-generating device, according to one or more example embodiments.

FIGS. 21-24 illustrate control methods for unlocking an aerosol-generating device, according to one or more example embodiments.

FIGS. 25-26 illustrate control methods for transmitting verified personal data for unlocking an aerosol-generating device, according to one or more example embodiments.

### DETAILED DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” “attached to,” “adjacent to,” or “covering” another element or layer, it may be directly on, connected to, coupled to, attached to, adjacent to or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations or sub-combinations of one or more of the associated listed items.

It should be understood that, although the terms first, second, third, etc. may be used herein to describe various elements, components, regions, layers and/or sections, these elements, components, regions, layers, and/or sections should not be limited by these terms. These terms are only used to distinguish one element, component, region, layer, or section from another region, layer, or section. Thus, a first element, component, region, layer, or section discussed below could be termed a second element, component, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

While the term “same” or “identical” is used in description of example embodiments, it should be understood that some imprecisions may exist. Thus, when one element is referred to as being the same as another element, it should be understood that an element or a value is the same as another element within a desired manufacturing or operational tolerance range (e.g., ±10%).

When the terms “about” or “substantially” are used in this specification in connection with a numerical value, it is intended that the associated numerical value includes a manufacturing or operational tolerance (e.g., ±10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

Aerosol-generating devices may heat a material to a temperature sufficient to release constituents of the material and/or employ other aerosol-generating means to release constituents of the material, such as ultrasonic, piezo actuation, pneumatic dispersions, etc. Some aerosol-generating devices may be in a locked state to prevent unauthorized consumers from using the devices. Such devices may be unlocked with a mobile computing device via wireless communications. In many cases, the process for unlocking the devices is cumbersome and sometimes restricted due to particular restrictions associated with the mobile computing device.

The systems, aerosol-generating devices, and methods according to the present disclosure leverage one or more wireless communication protocols between the devices and other consumer-controlled devices (e.g., operator terminals, government-issued biometric identifications, payment cards, etc.) to unlock the aerosol-generating devices. Such techniques enable the unlocking of the aerosol-generating devices in a consumer-friendly manner for adult consumers (e.g., consumers of an appropriate or legal age of adulthood, such as 18 years old, etc.), while also providing increased security measures to prevent unauthenticated consumers from using the devices.

In some embodiments, near-field communication (NFC) may be used between the devices and operator terminals to unlock the aerosol-generating devices. For example, and as further explained herein, an aerosol-generating device may receive an encrypted pass from an operator terminal associated with an adult consumer, in which the encrypted pass optionally includes at least one control parameter for the aerosol-generating device. In such examples, the pass may be created if the consumer is appropriately authenticated, may be encrypted with a public key, and may be stored in a digital wallet application of the operator terminal or in another suitable location accessible by the operator terminal. Once received, the aerosol-generating device may decrypt the pass using a unique private key stored therein to retrieve the at least one optional control parameter for the aerosol-generating device. Then, in response to decrypting the pass, the aerosol-generating device may be unlocked and controlled based on the at least one optional control parameter in the received pass.

As such, in these examples, the aerosol-generating devices may be unlocked and controlled by leveraging NFC communication between the devices and operator terminals. For example, NFC communication provides simplified use and pairing between the devices and operator terminals, consumer-friendly interactions, and low power consumption, while also providing enhanced security of the aerosol-generating devices by preventing unauthenticated consumers from using the devices and requiring proximity between the operator terminals and devices. Further, NFC communication may be leveraged to control the aerosol-generating devices by enabling the passage of control parameters in a secure data stream from the operator terminals to the aerosol-generating devices. In doing so, firmware settings in the aerosol-generating devices may be altered, thereby directing the devices to operate under custom control scenarios established by authenticated consumers.

Additionally, the systems, aerosol-generating devices, and methods herein may leverage the use of existing credentials to enable adult consumers to unlock and access the devices in a consumer-friendly manner, while also providing increased security measures to prevent unauthenticated consumers from using the devices. The existing credentials may include, for example, a government-issued digital identification (e.g., a mobile identification, such as a mobile driver's license (mDL), a mobile passport, a mobile non-driver's license, etc.) containing verified customer data and a physical document, such as a government-issued biometric identification (e.g., a biometric passport, etc.) containing verified customer data, a payment card (e.g., a bank card, etc.) containing verified customer data, etc. In such examples, the government-issued digital identification can be accessed through government-regulated applications containing the digital identifications or verified customer data corresponding thereto. In some examples, a digital pass may be created based on an existing credential (e.g., a digital identification, etc.) and stored in a digital wallet application of an operator terminal (e.g., a smart phone, etc.) for wirelessly unlocking an aerosol-generating device. In other examples, an existing physical, biometric identification (ID) or payment card can be used to wirelessly unlock an aerosol-generating device without necessary interaction with an operator terminal.

Using existing credentials to unlock the aerosol-generating devices provides a simplified, consumer-friendly approach for adult consumers to access the devices. For example, the aerosol-generating devices may be unlocked through an existing credential and any suitable wireless communication, such as NFC, Bluetooth® communication, Wi-Fi communication, and radio frequency identification (RFID) communication. Such wireless communication techniques can provide simplified use and pairing, consumer-friendly interactions, and low power consumption, while also providing enhanced security of the aerosol-generating devices by preventing unauthenticated consumers from using the devices. Additionally, and as further explained herein, the wireless communication techniques may be leveraged to control the aerosol-generating devices by enabling the passage of control parameters in a secured data stream to the aerosol-generating devices. In doing so, firmware settings in the aerosol-generating devices may be altered, thereby directing the devices to operate under custom control scenarios established by authenticated consumers.

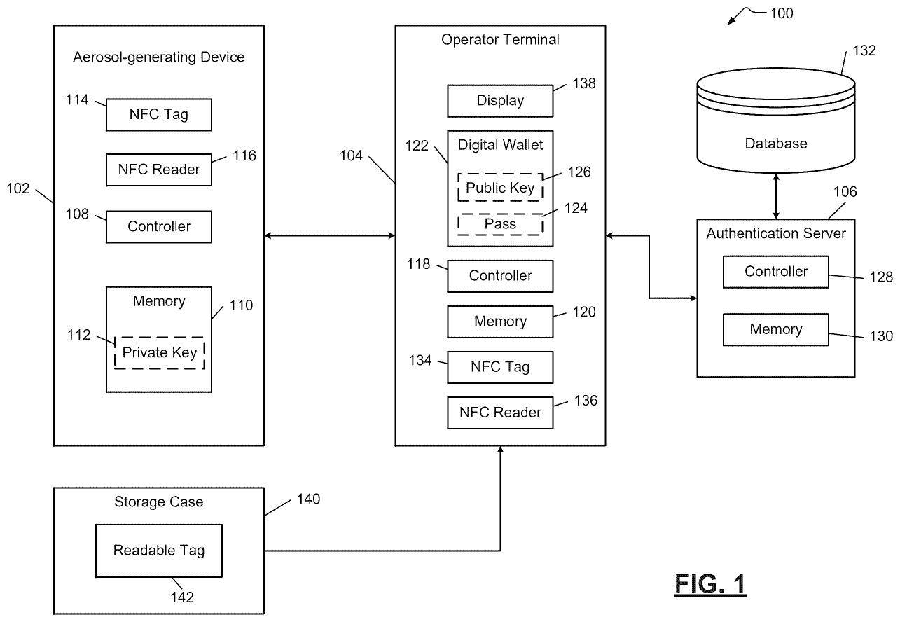

FIG. 1 illustrates an example system 100 for unlocking an aerosol-generating device 102 with an operator terminal 104 via NFC. As shown in FIG. 1, the system 100 generally includes the aerosol-generating device 102, the operator terminal 104, and an authentication server 106. In this example, the operator terminal 104 is in wireless communication with the aerosol-generating device 102 and the authentication server 106.

As shown in FIG. 1, the aerosol-generating device 102, the operator terminal 104, and the authentication server 106 each includes a controller and a memory. Specifically, the aerosol-generating device 102 includes a controller 108 and a memory 110, the operator terminal 104 includes a controller 118 and a memory 120, and the authentication server 106 includes a controller 128 and a memory 130. In various embodiments, each controller 108, 118, 128 may include processing circuitry such as hardware including logic circuits, a hardware/software combination such as a processor executing software, or a combination thereof. For example, the processing circuitry may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc. Additionally, each memory 110, 120, 130 may be a non-volatile memory storing computer-readable instructions for the respective controller 108, 18, 128 to perform algorithms described herein. As an example, any one of or each memory 110, 120, 130 may be an electrically erasable programmable read-only memory (EEPROM), such as a flash memory or the like.

In the example of FIG. 1, the aerosol-generating device 102 further includes an NFC tag 114 and an NFC reader 116. In such examples, the NFC tag 114 may be a passive component that uses electromagnetic fields to receive power from a nearby NFC reader, such as an NFC reader in another device that reads the NFC tag 114. When an NFC reader is placed near the NFC tag 114 (e.g., within 4 centimeters, within 5 centimeters, etc.), the NFC tag 114 can wirelessly transmit data from the aerosol-generating device 102 to the NFC reader. The NFC reader 116 is an active component that can wirelessly communicate (e.g., bidirectionally) with an NFC tag of another device. For example, the NFC reader 116 can read (e.g., receive) data from an NFC tag and write (e.g., transmit) data to an NFC tag, etc. In various embodiments, the NFC tag 114 and/or the NFC reader 116 may be embedded in a body of the device 102, located within the body (e.g., on a circuit board, etc.), etc.

In various embodiments, the NFC tag 114 and the NFC reader 116 may be separate components (e.g., separate transceivers on the same or different chipset) of the aerosol-generating device 102 as generally shown in FIG. 1. In other examples, the NFC tag 114 and the NFC reader 116 may be implemented with a single chipset switching between a tag mode and a reader mode. In such examples, a single transceiver may be configured by software to function as a tag or a reader.

In various embodiments, the aerosol-generating device 102 may be any suitable device that heats a material therein to a temperature sufficient to release constituents of the material. As examples only, and as further explained herein, the aerosol-generating device 102 may be an electronic-vaping (“e-vaping”) device that heats a pre-vapor formulation, a heated tobacco device that heats an aerosol-forming substrate, etc. Additionally, in some examples, the aerosol-generating device 102 may be configured to receive a consumable designed to be depleted/expended via use. In such examples, the consumable may be removable from the aerosol-generating device 102 when desired. As examples only, the consumable may include a cigarette, a heat stick, a pod, a cartridge, a capsule, a volatilizable material that can be optionally contained in, for example, a cartridge or capsule, etc.

With continued reference to FIG. 1, the operator terminal 104 further includes a digital wallet application 122. The digital wallet application 122 may generally store information relating to payment cards (e.g., credit cards, debit cards, gift cards, etc.), bank accounts, event tickets, validated information (e.g., a government ID such as a driver license, a passport, etc.), etc. As examples only, the digital wallet application 122 may be the Apply Pay® mobile payments solution, the Google Wallet™ digital wallet, the Samsung Pay® digital wallet, etc. Then, when desired, the digital wallet application 122 may be accessed for transactions, etc.

In various embodiments, the operator terminal 104 further includes an NFC tag 134, an NFC reader 136, and a display 138. In such examples, the NFC tag 134 and the NFC reader 136 of the operator terminal 104 may function in a similar manner as the NFC tag 114 and the NFC reader 116 of the aerosol-generating device 102 explained above. Additionally, the NFC tag 134 and the NFC reader 136 may be separate components (e.g., separate transceivers on the same or different chipset) of the operator terminal 104 (as generally shown in FIG. 1) or may be implemented with a single chipset switching between a tag mode and a reader mode. In such examples, a single transceiver may be configured by software to function as a tag or a reader. The display 138 may be any suitable device that is configured to render content including, for example, interfaces (e.g., interactive user interfaces, etc.), etc.

The operator terminal 104 may be any suitable computing device associated with an adult consumer. For example, the operator terminal 104 may be a mobile or static computing device associated with and generally controlled by the adult consumer. The mobile computing device may include, for example, a smart phone, a tablet, a laptop, a wearable device (e.g., a watch such as a smart watch paired to a smart phone, etc.) etc. In such examples, the smart phone may be any suitable type, such as Apple® iPhone® devices, Android™ based devices, etc.

In various embodiments, a key pair may be created for the aerosol-generating device 102. The key pair includes a set of cryptographic keys, such as a public key 126 used to encrypt data and a private key 112 used to decrypt data. In such examples, the private key 112 is a secret, mathematical key that is used in conjunction with the public key 126 to encrypt and decrypt data. The private key 112 may include a unique sequence of bits that are typically long and random or pseudo-random.

In such examples, the key pair may be specific to the aerosol-generating device 102 itself, a collection of aerosol-generating devices, a verified adult consumer, and/or the operator terminal 104. For example, the key pair may be created based on a device specific identifier for the device 102, one or more stock keeping units (SKUs) specific to a brand of aerosol-generating devices, based on consumer credentials, a specific identifier for the operator terminal 104, etc. As such, the key pair may be employed to unlock only the aerosol-generating device 102 and/or any one of a collection of aerosol-generating devices (e.g., all of which are owned by and tied to the adult consumer, tied to the operator terminal 104, etc.), as further explained below.

The key pair may be created during manufacturing of the device 102. In such examples, the private key 112 may be stored or otherwise programmed into the memory 110 of the aerosol-generating device 102 and the public key 126 may be provided to, for example, the authentication server 106. In other examples, the private key 112 and/or public key 126 may be created before manufacturing of the device 102 or created after manufacturing of the device 102 if desired.

In various embodiments, the public key 126 may be provided to the operator terminal 104 when, for example, the adult consumer is authenticated (e.g., age verified, etc.). This may be accomplished via an age verification process completed via the operator terminal 104. For example, the aerosol-generating device 102 may be purchased or otherwise obtained by the adult consumer. At this time, the aerosol-generating device 102 is in a locked state. Generally, the aerosol-generating device 102 may be in a locked state when manufactured or another time before the device 102 is purchased or obtained by the adult consumer. For instance, a power source in the device 102 may be disconnected (e.g., locked out) from a heater in the device 102. In other examples, a flag or bit may be set in the memory 110 of the device 102 to prevent the heater from operating.

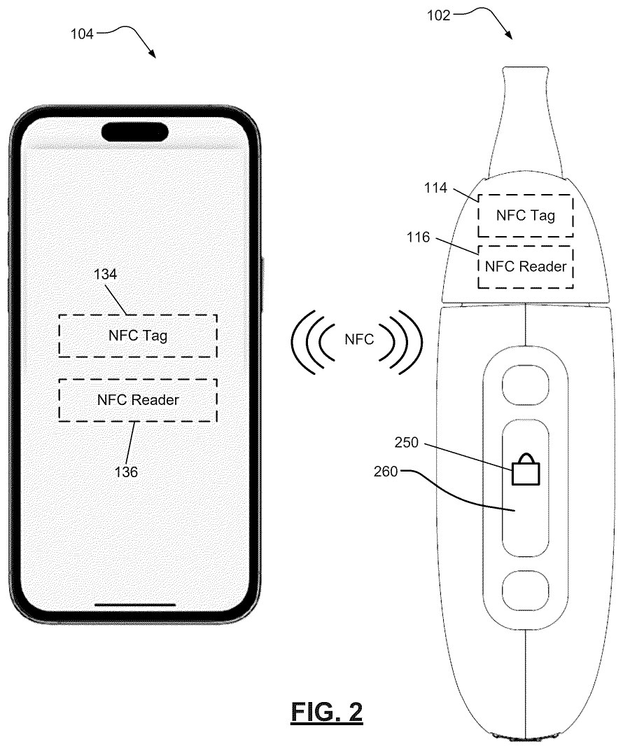

Then, the operator terminal 104 may be placed near the aerosol-generating device 102 to initiate the age verification process. For example, the NFC reader 136 of the operator terminal 104 may be placed near the NFC tag 114 (e.g., within 4 centimeters, within 5 centimeters, etc.). As one example, the operator terminal 104 may be placed in contact with (e.g., tapped against) the aerosol-generating device 102. For example, FIG. 2 depicts one example of this interaction between the operator terminal 104 and the aerosol-generating device 102 in a locked state. In this example, the operator terminal 104 (e.g., shown as a smart phone) is placed near the aerosol-generating device 102, to enable the NFC reader 136 to communicate with the NFC tag 114. Additionally, in some examples, the aerosol-generating device 102 may provide a notification of the locked state. For example, and as shown in FIG. 2, a display 260 of the aerosol-generating device 102 may provide an indicator 250, such as a locked padlock.

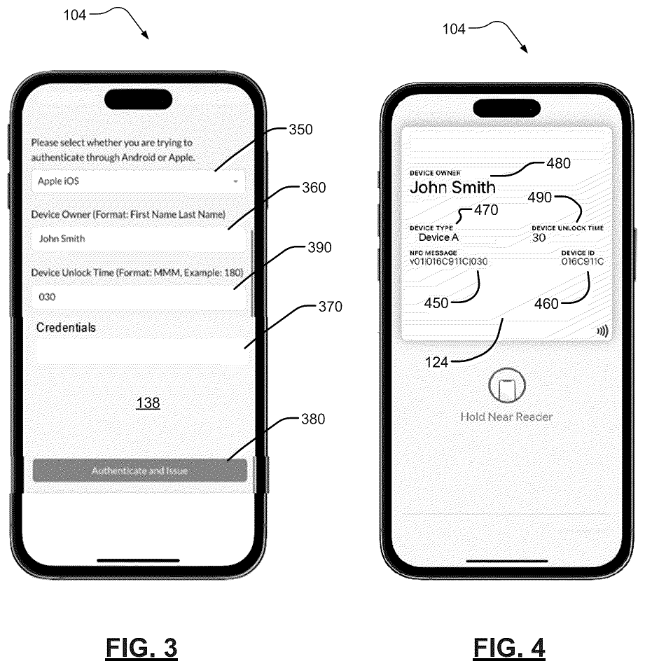

In response to detecting the NFC tag 114, the controller 118 may cause the operator terminal 104 to display an interactive user interface for authenticating the adult consumer. For example, the controller 118 may cause the display 138 to present a link to a website to register the device 102. Once selected, the interactive user interface may be presented by the display 138. In other examples, the controller 118 may cause the operator terminal 104 to automatically launch the website to present the interactive user interface. Then, the interactive user interface may be utilized to input consumer authenticating data. For example, the adult consumer may upload a picture of an identification (e.g., a driver's license, a passport, etc.), input a date of birth, social security number, and/or specific verifiable credentials of the adult consumer. Also, during this time, the adult consumer may select or otherwise provide other inputs, such as a specific operating system and/or device brand of the operator terminal 104, an owner of the device 102, etc. For example, FIG. 3 depicts one example of this interaction with the operator terminal 104. In this example, the display 138 of the operator terminal 104 (e.g., shown as a smart phone) renders the interactive user interface (e.g., via a website) with various inputs, including an input 350 for specifying an operating system of the operator terminal 104, an input 360 for identifying the owner of the device 102, and an input 370 for uploading or otherwise inputting consumer credentials. Once the necessary data is provided, the adult consumer may select an input 380 for transmitting the provided data to the authentication server 106.

In other examples, the operator terminal 104 may be placed near a storage case 140 for the aerosol-generating device 102 to initiate the age verification process. In such examples, the storage case (e.g., a device case, etc.) 140 may include a readable tag 142, such as an NFC tag, a QR code, etc. readable by the operator terminal 104 (e.g., by the NFC reader 136, a camera, etc.). In response to detecting the readable tag 142, the controller 118 may cause the operator terminal 104 to display an interactive user interface for authenticating the adult consumer, as explained above.

In still other embodiments, the age verification process may be completed via a point-of-sale terminal. For example, when the aerosol-generating device 102 is obtained by the adult consumer, the point-of-sale terminal may initiate the age verification process via input from the consumer or another individual (e.g., a salesperson). In such examples, the adult consumer may provide an identification (e.g., a driver's license, a passport, etc.), a phone number, a date of birth, etc. to the point-of-sale terminal and/or to an individual operating the point-of-sale terminal. Once the necessary data is provided, the point-of-sale terminal may transmit the provided data to the authentication server 106.

With continued reference to FIG. 1, the authentication server 106 may then authenticate the adult consumer, and more specifically the consumer credentials. For example, the authentication server 106 may verify the age of the adult consumer based on the consumer credentials and known credentials. For instance, the authentication server 106 may read the provided data, scrape the uploaded picture, etc. to obtain the consumer credentials (e.g., name, date of birth, address, etc.). Then, the authentication server 106 may request verified data from a database 132 for that consumer. In such examples, the database 132 may be a secured database (e.g., a third-party database) accessible by the authentication server 106. The authentication server 106 may then compare the provided consumer credentials with the verified data from the database 132. If the consumer credentials are valid and the consumer meets one or more thresholds (e.g., a defined age, a defined location, etc.), the consumer may be verified for using the aerosol-generating device 102. If the consumer credentials are invalid or the consumer does not meet one or more thresholds, the consumer may not be verified for using the aerosol-generating device 102.

If verified, the authentication server 106 proceeds with creating a pass (e.g. a payload pass) 124 specific to the authenticated adult consumer. In such examples, the pass 124 is provided to the operator terminal 104, along with the public key 126. Then, the operator terminal 104 may store the pass 124 and the public key 126 in the digital wallet application 122 as shown in FIG. 1, or in another suitable application of the operator terminal 104. For example, once created, the controller 118 may cause the display 138 to present a link to download the pass 124 and the public key 126 from the authentication server 106 to the digital wallet application 122 or another suitable application of the operator terminal 104. This link may be pushed by the authentication server 106 to the operator terminal 104 via, for example, the provided phone number. In such examples, the pass 124 may be also specific to the operator terminal 104. As such, the pass 124 may be only downable by the operator terminal 104 and/or not transferable to another operator terminal or another consumer.

In some examples, the pass 124 may be stored in another location, such as remotely from the operator terminal 104. For example, the pass 124 may be stored by another server and then accessible by the operator terminal 104 when desired. In such examples, the pass 124 may be used in conjunction with a web NFC protocol.

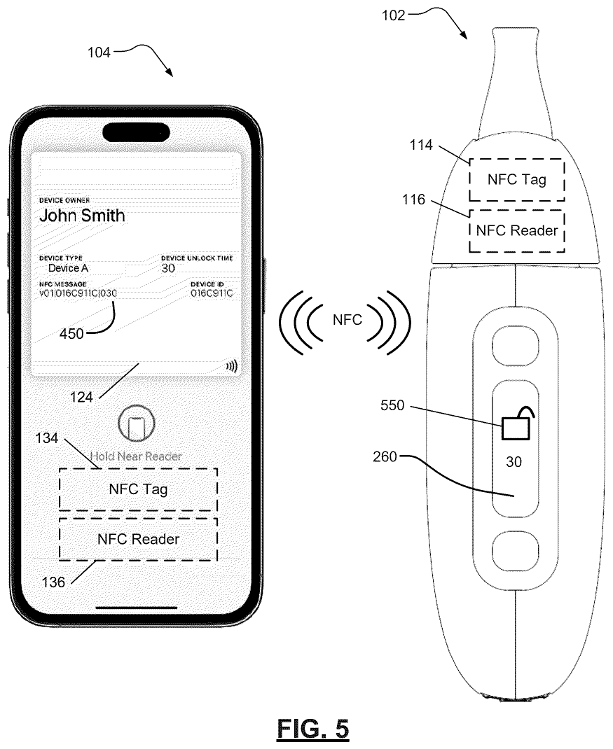

As one example, FIG. 4 depicts one example of the operator terminal 104 (e.g., shown as a smart phone) including the pass 124 stored in the digital wallet application 122 of FIG. 1. As shown, the pass 124 created by the authentication server 106 includes a string data message 450, an identifier 460 for the aerosol-generating device 102, a type or name 470 of the device 102, and a name 480 of the owner for the device 102. In such examples, the string data message 450 may include the identifier 460 along with other data that is passable to another device (as further explained below). In various embodiments, the identifier 460 may be initially provided to the authentication server 106 (along with the public key 126) before the pass 124 is created. In this example, while the identifier 460 specific to the aerosol-generating device 102 is shown as “016C911C,” it should be appreciated that any suitable identifier may be employed.

In various embodiments, the pass 124 may include one or more control parameters for the aerosol-generating device 102. In such examples, at least one control parameter may be input via the interactive user interface, the point-of-sale terminal, etc. during the age verification process explained above. With this configuration, the control parameter(s) may be provided to the authentication server 106 during the age verification process. In other examples, the control parameter(s) may be provided and/or updated after the age verification process.

In such examples, the control parameter(s) may be user defined settings. As an example, the control parameter(s) may include an unlock interval setting specifying a condition for transitioning back to a locked state. For example, the unlock interval setting may refer to a defined period of time, a defined number of puffs, an amount consumed, etc. As examples only, the defined period of time may be no more than 30 minutes, no more than 10 minutes, no more than 5 minutes, etc. For example, the interactive user interface of the operator terminal 104 in FIG. 3 is shown as including an input 390 for providing an unlock time of 30 minutes. As such, in this example, the pass 124 includes a device unlock time control parameter 490 specifying an unlock time of 30 minutes. In other examples, the unlock interval setting may be the removal and/or completion of a consumable (e.g., a pod, a cartridge, etc.) from the device 102. If this condition is met (e.g., removal and/or completion of the consumable is detected, etc.), the device 102 may transition to the locked state.

In still other examples, the control parameter(s) may include other optional settings, such as a temperature setting, a puff profile, a power setting, an interface setting, a consumer notification setting, etc. for the aerosol-generating device 102. In such examples, the temperature setting may include a preferred temperature setpoints selected by the adult consumer for heating an aerosol-forming substrate, a pre-vapor formulation, etc. The puff profile may enable the adult consumer to select from a table of heating profiles pre-configured on the device 102 (e.g. a tobacco setting, a menthol setting, etc.). The power setting may allow the adult consumer to select from power profiles having different setpoints. The interface setting (e.g., human machine interface settings) may allow the adult consumer to select different options for the display 260 of FIG. 2. The consumer notification setting may allow the consumer to select different alert configurations, such as different haptic settings, etc.

Then, once the adult consumer is verified as explained above, the authentication server 106 creates the pass 124 with the provided control parameter(s) therein. As such, when the pass 124 is provided to the operator terminal 104 and/or another location accessible by the operator terminal 104, so too are the specific control parameter(s) for the device 102. Such control parameter(s) for the device 102 may be included with the pass 124 when it is stored in the digital wallet application 122 and/or in the other accessible location. For example, the pass 124 of FIG. 4 is shown as including the control parameter (e.g., a 30 minute unlock time) provided via the input 390 of FIG. 3. Additionally, the string data message 450 also includes the specific control parameter(s) for the device 102 (e.g., “030”).

If the control parameter(s) are provided and/or updated after the age verification process, the authentication server 106 may generate an updated pass similar to the pass 124 but with the new control parameter(s) and a new string data message based on the new control parameter(s). In such examples, the pass 124 may maintain the same device credentials, consumer credentials, etc.

With continued reference to FIG. 1, once the pass 124 is created and stored in the digital wallet application 122 and/or in the other accessible location, the operator terminal 104 may initiate interaction with the aerosol-generating device 102 to unlock the device 102. In this example, the adult consumer may be required to take active steps to initiate the interaction. For example, the pass 124 may be required to be activated before use. In such examples, the pass 124 may be activated in response to user input. For instance, the adult consumer may select the pass 124 in the digital wallet application 122, select an activate input associated with the pass 124, etc. Once the pass 124 is activated (e.g., is active), the pass 124 may be utilized to unlock the device 102 as further explained below. In various embodiments, the pass 124 may become deactivated (e.g., time out) if not used within a period of time of being activated. This time out period may be a user defined setting associated with the digital wallet application 122.

In various embodiments, the operator terminal 104 may then be placed near the aerosol-generating device 102 to initiate unlocking of the device 102. For example, the operator terminal 104 may be placed within 4 centimeters, within 5 centimeters, etc. of the device 102. As one example, the operator terminal 104 may be placed in contact with (e.g., tapped against) the aerosol-generating device 102. With this arrangement, the operator terminal 104 may detect the aerosol-generating device 102. More specifically, if the operator terminal 104 and the aerosol-generating device 102 are within a sufficient distance apart, the NFC reader 136 of the operator terminal 104 may detect the NFC tag 114 of the device 102.

In other examples, the aerosol-generating device 102 may detect the operator terminal 104 in the vicinity of the device 102. When this occurs, the device 102 may transmit a signal (via NFC) to the operator terminal 104, thereby prompting the adult consumer to activate the pass 124 for use. In such examples, the controller 118 may generate a notification indicating the device 102 is nearby and ask if the adult consumer wants to proceed. Then, the adult consumer may provide input (e.g., tap on the notification, open an application, etc.) to open the pass 124 for activation as explained above.

In various embodiments, the NFC tag 114 of the aerosol-generating device 102 may be detectable only if one or more conditions are met. For example, in some embodiments the NFC tag 114 may be detectable (e.g., by the NFC reader 136 and/or other NFC readers) only if the aerosol-generating device 102 is in a locked state, such as shown in FIG. 5. In such examples, the NFC tag 114 may be active (e.g., detectable) when the device 102 is in the locked state. Conversely, if the device 102 is in an unlocked state, the NFC tag 114 may be inactive (e.g., undetectable). With this configuration, a power source associated with the device 102 may provide power to the NFC tag 114 and/or the NFC reader 136 only when the device 102 is in the locked state, thereby preserving power of the power source.

For example, FIG. 5 depicts one example of an interaction between the operator terminal 104 and the aerosol-generating device 102. In this example, the operator terminal 104 (e.g., shown as a smart phone) is placed near the aerosol-generating device 102, to enable the NFC reader 136 to detect the NFC tag 114. As shown, the operator terminal 104 includes the stored pass 124 having the string data message 450 of FIG. 4.

Once the aerosol-generating device 102 is detected, the operator terminal 104 may encrypt the pass 124 using the public key 126. For example, the digital wallet application 122 may use the public key 126 to scramble, change, etc. the string data message 450 of FIG. 5. In other examples, the digital wallet application 122 may utilize another encryption method, such as implementing a hash function (e.g., with or without being salted) if desired.

Then, once encrypted, the controller 118 of the operator terminal 104 causes the operator terminal 104 to transmit the encrypted pass 124 to the aerosol-generating device 102. For example, the controller 118 may enable or activate the NFC tag 134 once the pass 124 is encrypted. In such examples, the NFC reader 116 of the aerosol-generating device 102 may retrieve or otherwise receive the encrypted pass 124 from the NFC tag 134 of the operator terminal 104.

The aerosol-generating device 102 may then determine whether the pass 124 is valid and if so, unlock. For instance, to unlock the aerosol-generating device 102, the controller 108 may connect the power source and the heater in the device 102. In other examples, a locking flag or bit set in the memory 110 may be cleared, thereby allowing the heater to receive power. In still other examples, a flag or bit may be set in the memory 110 of the device 102 to allow the heater to receive power. In some examples, the aerosol-generating device 102 may provide a notification of the unlocked state. For example, and as shown in FIG. 5, the display 260 of the aerosol-generating device 102 may provide an indicator 550, such as an unlocked padlock.

The aerosol-generating device 102 may determine whether the pass 124 is valid in different manners. For example, the controller 108 may retrieve the private key 112 from the memory 110 in the aerosol-generating device 102, and then attempt to decrypt the received pass 124 using the private key 112. More specifically, the controller 108 attempts to decrypt the string data message 450 using the private key 112. In some examples, if the controller 108 can decrypt the string data message 450, the pass 124 is valid. Then, in response to decrypting the string data message 450, the controller 108 may unlock the aerosol-generating device 102. As such, in this example, the controller 108 may automatically unlock the aerosol-generating device 102 in response to decrypting the pass 124.

In other examples, the aerosol-generating device 102 may take additional steps to ensure the pass 124 is valid. For example, after the string data message 450 is decrypted with the private key 112, the controller 108 may retrieve the identifier 460 (or, for example, a SKU, etc.) from the message 450. Then, the controller 108 may compare the retrieved identifier 460 with a device identifier stored in the memory 110. In such examples, the device identifier may be stored in the memory 110 before, during, and/or after manufacturing of the device 102. The controller 108 may then determine whether the received pass 124 (e.g., the received string data message 450) is valid based on the comparison. For instance, if the retrieved identifier 460 and the stored device identifier match, the pass 124 may be determined to be valid. Similar steps may be taken if, for example, the string data message 450 may include a SKU. As such, in this example, the controller 108 may unlock the aerosol-generating device 102 in response to decrypting the pass 124 and determining that the pass 124 is valid.

In various embodiments, the aerosol-generating device 102 may be operated based on the control parameter(s) after the device 102 is unlocked. For example, and as explained above, the pass 124 may be created with user defined control parameter(s), such as an unlock interval setting, a temperature setting, a puff profile, a power setting, an interface setting, a consumer notification setting, etc. In such examples, the string data message 450 provided to the aerosol-generating device 102 may include each of the control parameter(s) for the device 102. Thus, when the string data message 450 is decrypted, the controller 108 may retrieve and store the control parameter(s) for the device 102.

Then, the controller 108 may control the aerosol-generating device 102 based on at least one of the received control parameter(s). For instance, if an unlock interval setting is received, the controller 108 may unlock the aerosol-generating device 102 until one or more conditions associated with the unlock interval setting are met. Then, the controller 108 may automatically lock the device 102. For example, the unlock interval setting may be a period of 30 minutes, as indicated on the display 260 of FIG. 5 or another suitable period. In this example, the controller 108 locks the device 102 in response to the period (e.g., 30 minutes, etc.) expiring.

In other examples, the controller 108 may not automatically lock the aerosol-generating device 102 after the interval setting (e.g., a period). Instead, the controller 108 may lock the device 102 in response to the period (e.g., 30 minutes, etc.) expiring and a heater in the device 102 being inactive. In such examples, if the heater is active in the device 102 (e.g., in use), the controller 108 waits until the next time the heater is inactive before locking the device 102, thereby preventing a negative consumer experience.

In still other examples, the unlock interval setting may be related to the removal of a consumable (e.g., a pod, a cartridge, etc.) from the aerosol-generating device 102. For example, the controller 108 may detect if a consumable is removed from the device 102. If the device 102 is in an unlocked state, the controller 108 may automatically lock the device 102 in response to the detected removal. In other examples, the controller 108 may detect when a new consumable is received, and then automatically lock the device 102 in response. In either case, the aerosol-generating device 102 is in a locked state when a new consumable is received.

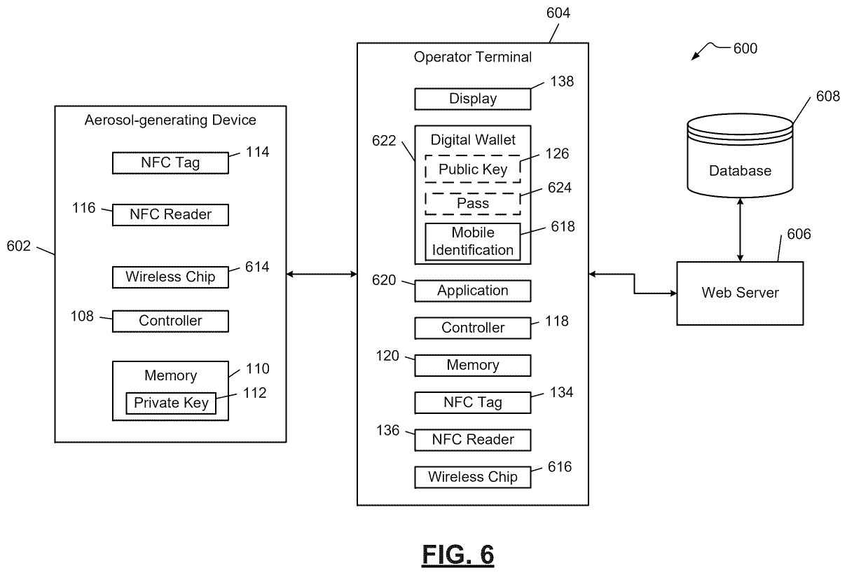

FIG. 6 illustrates an example system 600 for unlocking an aerosol-generating device 602 with an operator terminal 604 through the use of at least one existing credential unique to an adult consumer. As shown in FIG. 6, the system 600 generally includes the aerosol-generating device 602, the operator terminal 604, and a web server 606 external to the aerosol-generating device 602 and the operator terminal 604. In this example, the operator terminal 604 is in wireless communication with the aerosol-generating device 602 and the web server 606.

The aerosol-generating device 602 of FIG. 6 is similar to the aerosol-generating device 102 of FIG. 1. For example, the aerosol-generating device 602 of FIG. 6 includes the controller 108, the memory 110 storing the private key 112, the NFC tag 114 and the NFC reader 116 of FIG. 1, all of which are described above. Additionally, the aerosol-generating device 602 may include one or more wireless communication chips, such as a wireless communication chip 614 having a wireless protocol capability different than NFC. For instance, the wireless communication chip 614 may be a Bluetooth® chip, a Wi-Fi chip, etc. for communicating with the operator terminal 604 and/or another suitable device.

Additionally, the operator terminal 604 of FIG. 6 is similar to the operator terminal 104 of FIG. 1. For example, the operator terminal 604 of FIG. 6 includes the controller 118, the memory 120, the NFC tag 134, the NFC reader 136 and the display 138 of FIG. 1, all of which are described above. Additionally, the operator terminal 604 may include one or more wireless communication chips, such as a wireless communication chip 616 having a wireless protocol capability different than NFC. For instance, the wireless communication chip 616 may be a Bluetooth® chip, a Wi-Fi chip, etc. for communicating with the aerosol-generating device 602, the web server 606, and/or another suitable device.

In FIG. 6, the operator terminal 604 further includes a digital wallet application 622 similar to the digital wallet application 122 of FIG. 1. For example, the digital wallet application 622 may optionally include a pass 624 specific to the authenticated consumer and the public key 126 of FIG. 1. In such examples, the pass 624 may be similar to the pass 124 of FIG. 1. In this example, the authentication server 106 of FIG. 1 and/or another suitable consumer authenticating device can create and/or provide the pass 624 and the public key 126 to the operator terminal 604, as explained above.

Additionally, the digital wallet application 622 includes an existing credential, such as a validated ID unique to the adult consumer. For instance, in FIG. 6, the digital wallet application 622 includes a mobile identification 618 containing verified personal data and obtained from, for example, a government-controlled source. In such examples, the mobile identification 618 may include, for example, a mDL, a mobile passport, a mobile non-driver's license or any other suitable digital version of a government-issued identification. In other examples, the digital wallet application 622 may include a proprietary ID containing verified personal data and obtained from, for example, a private source capable of identity and age verification.

In various examples, the operator terminal 604 can be used to initiate one or more events to retrieve the mobile identification 618 from the government-controlled source and/or the proprietary ID from the private source. This retrieval may be in response to the NFC reader 136 (or another suitable wireless reader) of the operator terminal 604 detecting a wireless tag associated with the aerosol-generating device 602. For instance, the operator terminal 604 may be placed near a storage case (e.g., the storage case 140 of FIG. 1) for the aerosol-generating device 602. In this example, the storage case (e.g., a device case, etc.) may include a wireless tag, such as an NFC tag, a QR code, etc. readable by the operator terminal 604 (e.g., by the NFC reader 136, a camera, etc.). In other examples, the operator terminal 604 may be placed near the aerosol-generating device 602 so that the operator terminal 604 can read the NFC tag 114 (or another suitable wireless tag) of the device 602. When the wireless reader detects the wireless tag of the storage case and/or the aerosol-generating device 602, the controller 118 may initiate any one of different events to retrieve the mobile identification 618.

In some examples, the operator terminal 604 may retrieve the mobile identification 618 and/or the proprietary ID from the web server 606. For example, in response to detecting the wireless tag, the controller 118 may cause the operator terminal 604 to launch a web browser at a government-regulated website, a government-regulated application, and/or a proprietary application installed on the operator terminal 604 to communicate with the web server 606 and access the mobile identification 618 and/or the proprietary ID. In doing so, the controller 118 may generate a command to launch the web browser, the government-regulated application, or the proprietary application, any one of which can include an interactive user interface. The adult consumer may then utilize the interactive user interface on the display 138 to input credentials, such as consumer authenticating data.

For example, the adult consumer may upload a picture of an identification (e.g., a driver's license, a passport, etc.), input a date of birth, input a social security number, and/or input specific verifiable credentials of the adult consumer. This information may be sent to the web server 606 (e.g., via an application programming interface (API)), where the web server 606 may authenticate the adult consumer based on customer data in a database 608 or pass the credentials to another suitable device (e.g., the authentication server 106 of FIG. 1) for authenticating the adult consumer. Then, once authenticated, the web server 606 can provide the mobile identification 618 and/or the proprietary ID to the operator terminal 604 (e.g., through the government-regulated website, a government-regulated application, and/or a proprietary application). In turn, the controller 118 can store the mobile identification 618 and/or the proprietary ID containing verified personal data in the digital wallet application 622.

Then, the aerosol-generating device 602 may be unlocked through the use of the stored mobile identification 618 and/or the proprietary ID. For example, the controller 108 of the aerosol-generating device 602 may receive from the operator terminal 604, the verified personal data (or a portion or representative thereof) contained in the mobile identification 618 and/or the proprietary ID. In such examples, the verified personal data may be transmitted by the operator terminal 604 via any suitable wireless communication protocol, such as NFC, Bluetooth® , etc.

For example, once the adult consumer is authenticated, the pass 624 may be created (e.g., by the web server 606, the authentication server 106, etc.) and provided to the operator terminal 604. In such examples, the pass 624 may include the verified personal data from the mobile identification 618 and/or the proprietary ID. In some examples, the pass 624 may be encrypted with the public key 126, as explained above.

Then, the pass 624 with the verified personal data can be transmitted to the device 602 via the NFC tag 134 in the operator terminal 604 and the NFC reader 116 in the aerosol-generating device 602. In some examples, this transmission of the pass 624 (or more specifically, the verified personal data) can be initiated in response to the NFC reader 136 (or another suitable wireless reader) of the operator terminal 604 detecting a wireless tag associated with the aerosol-generating device 602, as explained above. For instance, when the NFC reader 136 detects the NFC tag 114 of the aerosol-generating device 602, the controller 118 may launch (e.g., open, etc.) the digital wallet application 622 for initiating transmission of the pass 624 containing the verified personal data. In such examples, the display 138 of the operator terminal 604 may provide a selectable input requesting the adult consumer to confirm this transmission. Once the digital wallet application 622 is launched, the controller 118 can transmit the pass 624 to the aerosol-generating device 602 via the NFC tag 134 of the operator terminal 604 and the NFC reader 116 of the aerosol-generating device 602.

In other examples, the verified personal data from the mobile identification 618 and/or the proprietary ID may be transmitted to the aerosol-generating device 602 via the wireless communication chip 616 (e.g., a Bluetooth® chip) in the operator terminal 604 and the wireless communication chip 614 (e.g., a Bluetooth® chip) in the aerosol-generating device 602. With this approach, the operator terminal 604 may be used to initiate a wireless pairing between the operator terminal 604 and the aerosol-generating device 602. For instance, when a wireless reader of the operator terminal 604 detects a wireless tag of the aerosol-generating device 602, the controller 118 may initiate a Bluetooth® wireless pairing between the operator terminal 604 and the aerosol-generating device 602. Alternatively, the adult consumer can provide an input to the operator terminal 604 to initiate a Bluetooth® wireless pairing between the operator terminal 604 and the aerosol-generating device 602.

In either case, the verified personal data (or a portion or representative thereof) can be transmitted to the aerosol-generating device 602 via the Bluetooth® wireless pairing. For example, after the operator terminal 604 and the aerosol-generating device 602 are wirelessly paired, the controller 118 or the wireless communication chip 616 may generate a data packet including a bit value signifying the adult consumer is verified for using the aerosol-generating device 602, portions of the verified personal data (e.g., data representing the adult consumer's date of birth, etc.), etc. Then, the data packet may be sent via the Bluetooth® wireless communication protocol to the wireless communication chip 614 of the device 602.

This transmission of the verified personal data to the aerosol-generating device 602 via the Bluetooth® wireless pairing may originate through the digital wallet application 622 or another application installed on the operator terminal 604. For example, the operator terminal 604 may include an application 620, such as a progressive web application (PWA) for communicating with the aerosol-generating device 602. In such examples, the application 620 may be used for unlocking the aerosol-generating device 602. For example, the application 620 may access the verified personal data (e.g., in the mobile identification 618 and/or the proprietary ID) stored in the digital wallet application 622 via an API. Then, once the application 620 has access to this verified credential, the operator terminal 604 can use the application 620 to transmit the data to the device 602 via the wireless communication chip 616 (e.g., a Bluetooth® chip) in the operator terminal 604 and the wireless communication chip 614 (e.g., a Bluetooth® chip) in the aerosol-generating device 602, as explained above. In such examples, the application 620 may provide a selectable input on the display 138 requesting the adult consumer to confirm or initiate this transmission.

Then, through any one of the wireless transmissions explained above, the controller 108 of the aerosol-generating device 602 can receive the verified personal data (or a portion or representative thereof). For example, the aerosol-generating device 602 can obtain the verified personal data from the pass 624. In such examples, if the pass 624 is encrypted, the controller 108 can decrypt the pass 624 with the private key 112 and retrieve the verified personal data from the pass 624. In other examples, the controller 108 can obtain the verified personal data from the received data packet.

Once the verified personal data is received, the controller 108 can determine whether the adult consumer meets an age condition based on this data. For example, the verified personal data may include a bit value signifying the adult consumer is verified for using the aerosol-generating device 602 and/or data representing the adult consumer's date of birth, data representing the adult consumer's age, etc. In such examples, the controller 108 can determine whether the adult consumer meets an age condition by recognizing a bit value is true (e.g., is high, etc.), determining the age of the adult consumer is greater than a threshold, etc. If the controller 108 determines that the adult consumer meets an age condition, the controller 108 can unlock the aerosol-generating device 602, as explained herein. For instance, to unlock the aerosol-generating device 602, the controller 108 may connect a power source and a heater in the device 602, clear a locking flag or bit set in the memory 110, set a flag or bit in the memory 110, etc.

Additionally, in some examples, the application 620 may be used for configurating the aerosol-generating device 602. For instance, the operator terminal 604 can use the application 620 to transmit one or more control parameters for the aerosol-generating device 602 via the wireless communication chip 616 in the operator terminal 604 and the wireless communication chip 614 in the aerosol-generating device 602. With this approach, the adult consumer may input some or all of the control parameters via an interactive user interface associated with the application 620. In such examples, the control parameter(s) may relate to any suitable setting (e.g., user defined settings, optional settings, etc.) for the configurating or controlling the aerosol-generating device 602, as explained above.

Further, the application 620 may be used for obtaining diagnostic data of the aerosol-generating device 602. For example, the application 620 may receive diagnostic data from the aerosol-generating device 602 via the wireless communication chip 616 in the operator terminal 604 and the wireless communication chip 614 in the device 602. In such examples, the diagnostic data may include, for example, usage data (e.g., usage times, etc.), battery level data (e.g., a remaining battery capacity, etc.), consumable data (e.g., a remaining consumable level, a material or materials in the consumable, etc.). The application 620 can then display such data on the interactive user interface associated with the application 620.

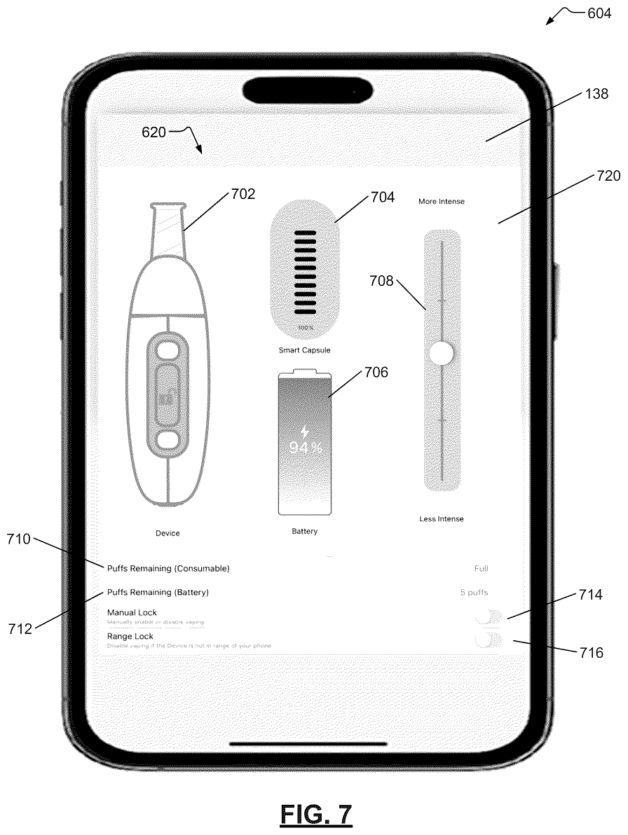

FIG. 7 depicts one example of the operator terminal 604 of FIG. 6 including the application 620, the display 138, and an interactive user interface 720 provided with the application 620 on the display 138. As shown in FIG. 7, the operator terminal 604 (e.g., shown as a smart phone) displays various consumer notifications about an aerosol-generating device, such as the aerosol-generating device 602 of FIG. 6. For instance, in FIG. 7, the interactive user interface 720 provides an image 702 of the aerosol-generating device, a remaining consumable level 704, a remaining capacity 706 of a battery in the aerosol-generating device, an intensity input 708 selectable by the adult consumer to modify an evaporation rate associated with the aerosol-generating device, a first puffs remaining notification 710 representing a number of consumer puffs remaining for the consumable associated with the aerosol-generating device, a second puffs remaining notification 712 representing a number of consumer puffs remaining until the battery in the aerosol-generating device depletes, a manual lock input 714 selectable by the adult consumer to lock the aerosol-generating device, and a range lock input 716 selectable by the adult consumer to lock the aerosol-generating device if the device is not within a defined range (e.g., a wireless communication range) of the operator terminal 604.

In some examples, any one of the aerosol-generating devices herein may be unlocked through the use of another existing credential unique to an adult consumer, such as a physical document having wireless communication capabilities. In such examples, the physical document may include a payment card issued to the adult consumer, a biometric ID issued to the adult consumer, etc.

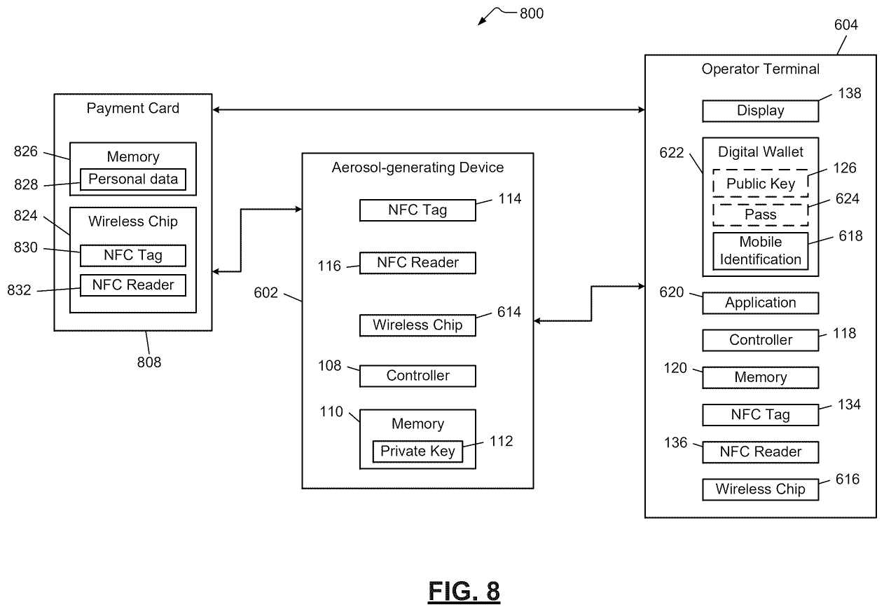

For example, FIG. 8 illustrates another example system 800 for unlocking the aerosol-generating device 602 of FIG. 6 through the use of a physical document having wireless communication capabilities and unique to an adult consumer. As shown in FIG. 8, the system 800 generally includes the aerosol-generating device 602 and the operator terminal 604 of FIG. 6, along with a payment card 808. In FIG. 8, the payment card 808 and the operator terminal 604 are in wireless communication with the aerosol-generating device 602. With this arrangement, the aerosol-generating device 602 may be unlocked in any one of the manners explained above relative to FIG. 6 and/or with the payment card 808 as further explained below.

While the system 800 of FIG. 8 is shown as including the aerosol-generating device 602 and the operator terminal 604 of FIG. 6, it should be appreciated that other example devices and/or operator terminals may be employed, such as the aerosol-generating device 102 and/or the operator terminal 104 of FIG. 1.

In the example of FIG. 8, the payment card 808 may be any suitable card having wireless communication capabilities and unique to the adult consumer. For example, the payment card 808 may be a debit card, a credit card, or any other suitable contactless bank card issued by a financial institution to the adult consumer, such as a bank, a credit union, a credit card issuer, etc. that handle financial and monetary transactions. In such examples, the payment card 808 is issued to the adult consumer following a verification process to authenticate the consumer.

As shown in FIG. 8, the payment card 808 generally includes a wireless communication chip 824 and a memory 826 to store verified personal data 828 of the adult consumer. The memory 826 may be, for example, an electrically erasable programmable read-only memory (EEPROM), such as a flash memory or the like. In some examples, the verified personal data 828 may include a bit value signifying the adult consumer is verified for using the aerosol-generating device 602 (e.g., age verified), data representing the adult consumer's date of birth, etc. In some examples, components of the wireless communication chip 824 and the memory 826 may be combined into a single chip in the payment card 808.

In FIG. 8, the wireless communication chip 824 may include a wireless tag and/or a wireless reader for communicating with other devices, such as the aerosol-generating device 602, the operator terminal 604, etc. The wireless tag and/or the wireless reader may be, for example, an NFC tag 830 and/or an NFC reader 832, as shown in FIG. 8. With this approach, the NFC tag 830 and the NFC reader 832 may be separate components (e.g., separate transceivers on the same chipset) in the payment card 808 (as shown) or a single component switching between a tag mode and a reader mode, as explained above. In other examples, the wireless communication chip 824 may be another suitable wireless communication device, such as a Bluetooth® chip.

In FIG. 8, the aerosol-generating device 602 includes the controller 108, the memory 110 storing the private key 112, the NFC tag 114, the NFC reader 116, and the wireless communication chip 614, all of which are described above. In this example, the wireless communication chip 614 is an optional component in the device 602. Additionally, the operator terminal 604 includes the controller 118, the memory 120, the NFC tag 134, the NFC reader 136, the display 138, the wireless communication chip 616, the application 620, and the digital wallet application 622, all of which are described above. The digital wallet application 622 includes the public key 126, the pass 624 specific to the authenticated adult consumer, and the mobile identification 618 (or another suitable form of verified personal data), all of which are described above. The wireless communication chip 616 and the application 620 are optional components in the device 602.

In the example of FIG. 8, the aerosol-generating device 602 may be unlocked through the use of the payment card 808. For example, the payment card 808 may be linked to the mobile identification 618 stored in the digital wallet application 622. In such examples, the adult consumer can select an input through the application 620 on the operator terminal 604 to link the payment card 808 and the mobile identification 618. With this approach, the application 620 can communicate the verified personal data associated with the mobile identification 618 to the payment card 808, which then stores the data (e.g., the verified personal data 828) in the memory 826. In this example, the operator terminal 604 and the payment card 808 can communicate via NFC through the NFC tag 134 and the NFC reader 136 of the operator terminal 604 and the wireless communication chip 824 in the payment card 808, or via Bluetooth® through the wireless communication chip 616 of the operator terminal 604 and a Bluetooth® wireless communication chip in the payment card 808.

Additionally, or alternatively, the payment card 808 may be linked to the mobile identification 618 stored in the digital wallet application 622 through the pass 624 (e.g., an NFC pass). In such examples, the pass 624 can be created specifically for the authenticated adult consumer, as explained above. With this approach, the pass 624 may include data representing the mobile identification 618 and be encrypted with the public key 126. In this example, the operator terminal 604 may communicate the pass 624 (e.g., the encrypted pass) to the payment card 808 via the NFC tag 134 and/or reader 136 of the operator terminal 604 and the wireless communication chip 824 in the payment card 808. Once received, the payment card 808 may store the pass 624 (e.g., the verified personal data 828) in the memory 826.

Then, the aerosol-generating device 602 can obtain the verified personal data 828 or a representation thereof from the payment card 808. For example, the verified personal data 828 may be transmitted to the device 602 via the wireless communication chip 824. In other examples, the aerosol-generating device 602 can obtain the pass 624 and retrieve data representing the mobile identification 618 from the pass 624 (e.g., by decrypting the pass 624 with the private key 112, etc.), as explained above. In either example, the verified personal data 828 may be received by the aerosol-generating device 602 when the payment card 808 is placed near or in contact with the aerosol-generating device 602, thereby enabling the NFC reader 116 of the aerosol-generating device 602 to detect the NFC tag 830 of the payment card 808 and initiate receipt of the verified personal data 828. In such examples, the proximity of the operator terminal 604 to the aerosol-generating device 602 is irrelevant as the payment card 808 itself is used to unlock the device 602. As such, ongoing use of the aerosol-generating device 602 is possible by only locating the payment card 808 near or in contact with the aerosol-generating device 602.

Once the data from the payment card 808 is received, the controller 108 can determine whether the adult consumer meets an age condition based on the received data, as explained above. If the controller 108 determines that the adult consumer meets an age condition based on the received data (e.g., a bit value is true, the age of the adult consumer is greater than a threshold, etc.), the controller 108 can unlock the aerosol-generating device 602. In such examples, to unlock the aerosol-generating device 602, the controller 108 may connect a power source and a heater in the device 602, clear a locking flag or bit set in the memory 110, set a flag or bit in the memory 110, etc.

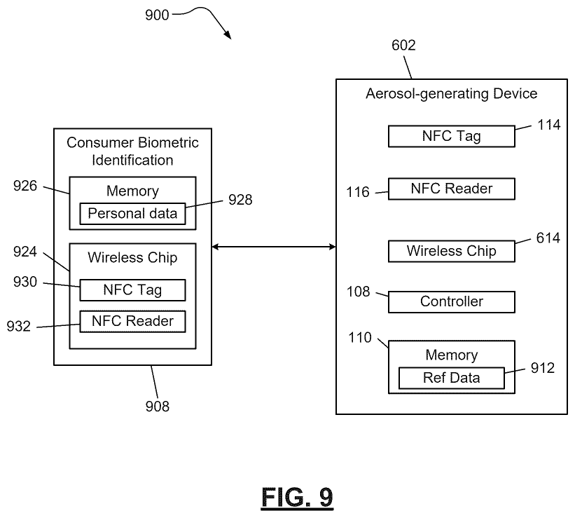

FIG. 9 illustrates another example system 900 for unlocking the aerosol-generating device 602 of FIG. 6 through the use of another physical document having wireless communication capabilities and unique to an adult consumer. Specifically, and as shown in FIG. 9, the system 900 generally includes the aerosol-generating device 602 of FIG. 6 and a biometric ID 908. In FIG. 9, the aerosol-generating device 602 may be in wireless communication with the biometric ID 908 and/or other devices, such as the operator terminal 604 of FIG. 6. With this arrangement, the aerosol-generating device 602 may be unlocked with the biometric ID 908 and/or in any one of the manners explained above relative to FIG. 6.

While the system 900 of FIG. 9 is shown as including the aerosol-generating device 602, it should be appreciated that other example aerosol-generating devices may be employed, such as the aerosol-generating device 102 of FIG. 1.

In the example of FIG. 9, the biometric ID 908 may be any suitable ID having wireless communication capabilities and unique to the adult consumer. For example, the biometric ID 908 may be any suitable government-issued document having wireless communication capabilities. For instance, the biometric ID 908 may be a biometric passport (e.g., a biometric passport book, a biometric passport card, etc.), a biometric driver's license, etc. issued by a government institution to the adult consumer following a verification process to authenticate the consumer.

As shown in FIG. 9, the biometric ID 908 generally includes a wireless communication chip 924 and a memory 926 to store verified personal data 928 of the adult consumer. The wireless communication chip 924 may be embedded into a page, a cover, etc. of a biometric passport book or into a plastic body of a biometric driver's license or passport card. The memory 926 may be, for example, an electrically erasable programmable read-only memory (EEPROM), such as a flash memory or the like. In some examples, components of the wireless communication chip 924 and the memory 926 may be combined into a single chip in the biometric ID 908.

In FIG. 9, the verified personal data 928 may include one or more pieces of information for identifying and verifying the adult consumer. For example, the verified personal data 928 may include identification information unique to the adult consumer, such as a name, a date of birth, a social security number, etc. Additionally, in some examples, the verified personal data 928 may include biometric information, such as a unique physical characteristic of the adult consumer. This unique physical characteristic may include, for example, a facial image, a fingerprint, etc. of the adult consumer.

The wireless communication chip 924 may include a wireless tag and/or a wireless reader for communicating with other devices, such as the aerosol-generating device 602, etc. The wireless tag and/or the wireless reader may be, for example, an NFC tag 930 and/or an NFC reader 932. With this approach, the NFC tag 930 and the NFC reader 932 may be separate components (e.g., separate transceivers on the same chipset) in the biometric ID 908 as shown in FIG. 9, or a single component switching between a tag mode and a reader mode, as explained above. In other examples, the wireless communication chip 924 may be another suitable wireless communication device, such as a Bluetooth® chip.

In FIG. 9, the aerosol-generating device 602 includes the controller 108, the memory 110, the NFC tag 114, the NFC reader 116, and the wireless communication chip 614, all of which are described above. In this example, the wireless communication chip 614 is an optional component in the device 602.

The aerosol-generating device 602 may be unlocked through the use of the biometric ID 908. For example, the aerosol-generating device 602 can obtain the verified personal data 928 or a representation thereof from the biometric ID 908. For instance, the verified personal data 928 may be transmitted to the device 602 via the wireless communication chip 924. More specifically, the verified personal data 928 may be received by the aerosol-generating device 602 when the biometric ID 908 is placed near or in contact with the aerosol-generating device 602, thereby enabling the NFC reader 116 of the aerosol-generating device 602 to detect the NFC tag 930 of the biometric ID 908 and initiate receipt of the verified personal data 928. In such examples, the biometric ID 908 itself may be used to unlock the device 602. As such, ongoing use of the aerosol-generating device 602 is possible by only locating the biometric ID 908 near or in contact with the aerosol-generating device 602.

Once the data from the biometric ID 908 is received, the controller 108 can determine whether the adult consumer meets an age condition based on the received data, as explained above. For instance, a biometric of the adult consumer meeting the age condition may be stored in the memory 110 and used as a reference (e.g., reference data 912 in FIG. 9) to compare with biometric information provided by the biometric ID 908. In such examples, the biometric of the adult consumer (e.g., a facial image, a fingerprint, etc.) may be established with an external device (e.g., the operator terminal 604 of FIG. 6) and provided to the device 602 for storing in the memory 110 or established with a biometric sensor (e.g., a fingerprint sensor, etc.) on the device 602. In other examples, identification information of the adult consumer meeting the age condition may be stored in the memory 110 and used as a reference (e.g., reference data 912 in FIG. 9) to compare with identification information provided by the biometric ID 908. With this approach, the adult consumer may provide the reference identification information to an external device (e.g., the operator terminal 604 of FIG. 6) during an authentication process. In turn, this reference identification information may be provided to the device 602 for storing in the memory 110.

If the controller 108 determines that the adult consumer meets an age condition based on the received data, the controller 108 can unlock the aerosol-generating device 602. In such examples, to unlock the aerosol-generating device 602, the controller 108 may connect a power source and a heater in the device 602, clear a locking flag or bit set in the memory 110, set a flag or bit in the memory 110, etc.

In various embodiments, the data (e.g., verified personal data stored in the payment card 808, verified personal data stored in the biometric ID 908, a created pass, etc.) used to unlock one aerosol-generating device may be generic to multiple aerosol-generating devices, such that that same data may be employed to unlock any one of multiple different aerosol-generating devices. For example, and with reference to FIGS. 1 and 6, the pass 124 may be generic to multiple aerosol-generating devices including the aerosol-generating devices 102, and the pass 624 may be generic to multiple aerosol-generating devices including the aerosol-generating devices 602. For instance, any of the passes herein may include a SKU specific to a collection of aerosol-generating devices. In such examples, the collection of aerosol-generating devices may include a similar brand of devices, aerosol-generating devices owned by and/or tied to the verified consumer, aerosol-generating devices tied to a particular operator terminal (e.g., the operator terminal 104), etc. With this configuration, a string data message in the pass may include the SKU or other identifying data for the collection of aerosol-generating devices.

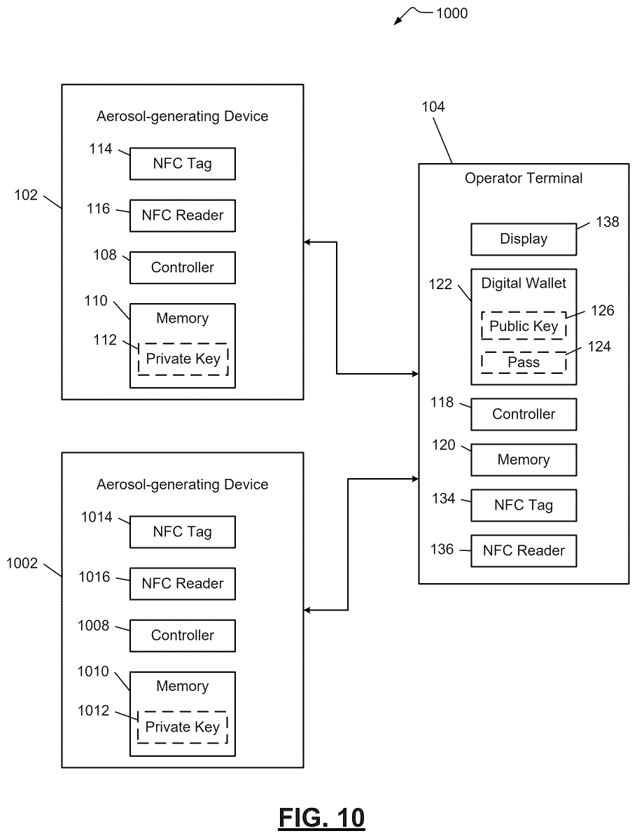

As one example, FIG. 10 depicts a system 1000 including the operator terminal 104 and the aerosol-generating device 102 of FIG. 1, and another aerosol-generating device 1002. While the system 1000 of FIG. 10 includes two aerosol-generating devices, it should be appreciated that more than two aerosol-generating devices may be employed.

As shown, the aerosol-generating device 1002 includes a controller 1008, a memory 1010, an NFC tag 1014, and an NFC reader 1016 which function similar to the controller 108, the memory 110, the NFC tag 114, and the NFC reader 116, respectively, of the device 102 explained above. In this example, the memory 1010 may include the same private key as the device 102 or a different private key 1012 that is able to decrypt the pass 124 (encrypted by the public key 126). In this example, the pass 124 may have a SKU specific to both devices 102, 1002, thereby enabling the pass 124 to unlock both devices.

Additionally, any one of the aerosol-generating devices herein may interact with and be unlocked by multiple operator terminals, multiple physical documents (e.g., payment cards, biometric IDs, etc.), etc.

For example, and with continued reference to FIG. 1, the aerosol-generating device 102 may interact with and be unlocked by multiple operator terminals, including the operator terminal 104. In such examples, each additional operator terminal may receive a pass similar to the pass 124 for unlocking the aerosol-generating device 102. For example, if the same consumer has multiple operator terminals (e.g., a smart phone, a smart watch, etc.), each operator terminal may include the same pass 124 or a substantially similar pass for unlocking the device. If the multiple operator terminals are owned by and/or controlled by different consumers that share the aerosol-generating device 102, each consumer may undergo the age verification process explained above, whereby a pass (e.g., the pass) is created and provided to or accessible by the respective operator terminal. Then, any one of the operator terminals may unlock the device 102 as explained above.

In other examples, and with continued reference to FIG. 8, the aerosol-generating device 602 may interact with and be unlocked by multiple payment cards, including the payment card 808. In such examples, each additional payment card may receive verified personal data (e.g., a pass similar to the pass 624) for unlocking the aerosol-generating device 602. For example, if the same consumer has multiple payment cards, each payment card may include the same verified personal data for unlocking the device. If the multiple payment cards are issued to different consumers that share the aerosol-generating device 602, each consumer may undergo the process explained above, whereby verified personal data (e.g., a pass) is linked to a respective operator terminal. Then, any one of the operator terminals may unlock the device 602 as explained above.

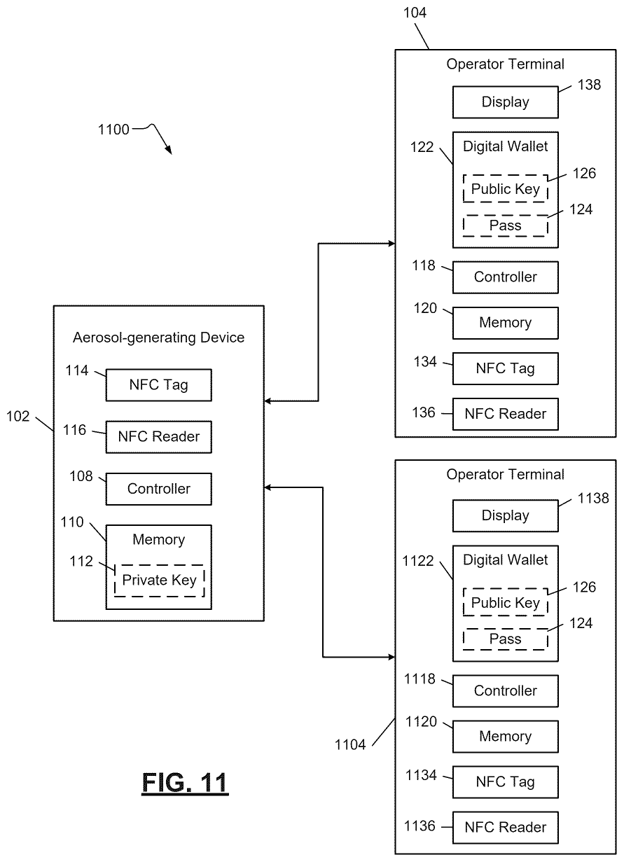

As one example, FIG. 11 depicts a system 1100 including the operator terminal 104 and the aerosol-generating device 102 of FIG. 1, and another operator terminal 1104. While the system 1100 of FIG. 11 includes two operator terminals, it should be appreciated that more than two operator terminals may be employed.

As shown, the operator terminal 1104 includes a controller 1118, a memory 1120, a digital wallet application 1122, an NFC tag 1134, an NFC reader 1136, and a display 1138 which function similar to the controller 118, the memory 120, the digital wallet application 122, the NFC tag 134, the NFC reader 136, and the display 138, respectively, of the operator terminal 104 explained above. In this example, the digital wallet application 1122 may include the same pass 124 and public key 126 as the operator terminal 104 or a different pass and/or a different public key.

Further, any one of the aerosol-generating devices herein may include one or more active ingredients, as further explained below. In such examples, one or more of the active ingredients in a particular aerosol-generating device may be selectively activated for consumption based on the data (e.g., verified personal data stored in the payment card 808, verified personal data stored in the biometric ID 908, a created pass, etc.) used to unlock the aerosol-generating device

For example, and with continued reference to FIG. 1, the aerosol-generating device 102 may include one or more active ingredients, such as nicotine, a cannabinoid (e.g., THC, etc.), an energizing agent (e.g., caffeine, etc.), etc. that is released and consumed by the adult consumer when the device 102 is used. For instance, the one or more active ingredients may be part of a consumable (e.g., a pod, a cartridge, etc.) receivable by the device 102 and the consumable may provide a signal to the controller 108 indicating the active ingredient(s) therein. In such examples, the pass 124 may enable activation of at least one active ingredient. For instance, during the age verification process (e.g., authentication process), the adult consumer may select one or more active ingredients via the interactive user interface on the operator terminal 104. In other examples, the authentication server 106 may select one or more active ingredients based on the consumer credentials (e.g., age, residence, etc.) received from the operator terminal 104. Then, when created, the pass 124 may contain one or more selected active ingredients (e.g., an identifier for each active ingredient). As such, when the pass 124 is decrypted, the controller 108 may retrieve the selected active ingredient(s) from the pass 124, enabling the controller 108 to activate only the selected active ingredient(s).

Additionally, a controller in one of the aerosol-generating devices herein may prevent activation of selected active ingredient(s) based on the data (e.g., verified personal data stored in the payment card 808, verified personal data stored in the biometric ID 908, a created pass, etc.) used to unlock the aerosol-generating device.

For example, and with continued reference to FIG. 1, the controller 108 may prevent activation of selected active ingredient(s). For instance, during the age verification process (e.g., authentication process), the adult consumer may select one or more restricted active ingredients. In other examples, the authentication server 106 may select one or more restricted active ingredients based on the consumer credentials (e.g., age, residence, etc.), ages of others in the consumer's household, etc. received from the operator terminal 104. When created, the pass 124 may contain one or more restricted active ingredients. Then, when the pass 124 is decrypted, the controller 108 may retrieve the restricted active ingredient(s) from the pass 124 and detect the active ingredient(s) in the consumable (e.g., based on a signal from the consumable), enabling the controller 108 to prevent activation of some or all of the active ingredient(s) in the consumable.

In addition, in some embodiments, the data (e.g., verified personal data stored in the payment card 808, verified personal data stored in the biometric ID 908, a created pass, etc.) used to unlock the aerosol-generating device may be required for only one or more selected active ingredients.

For example, the pass 124 of FIG. 1 may be required for only one or more selected active ingredients. In other words, some active ingredients (e.g., caffeine, etc.) may not be restricted and therefore may be consumed without the pass 124. In such examples, the controller 108 may automatically unlock the aerosol-generating device 102 when a consumable is received and detected with caffeine and/or other unrestricted ingredients (e.g., when the consumable provides a signal indicating the active ingredient(s) therein). However, if the aerosol-generating device 102 receives a consumable with another active ingredient (e.g., nicotine, a cannabinoid, etc.), the controller 108 may maintain the aerosol-generating device 102 in the locked state until the pass 124 is received, as explained above.

In various embodiments, any one of the aerosol-generating devices herein may be any suitable device that heats a material therein to a temperature sufficient to release constituents of the material. As examples only, any one of the aerosol-generating devices may be an electronic-vaping (“e-vaping”) device that heats a pre-vapor formulation, a heated tobacco device that heats an aerosol-forming substrate, etc. Additionally, in some examples, any one of the aerosol-generating devices may be configured to receive a consumable designed to be depleted/expended via use. In such examples, the consumable may be removable from the aerosol-generating device when desired. As examples only, the consumable may include a cigarette, a heat stick, a pod, a cartridge, a capsule, a volatilizable material that can be optionally contained in, for example, a cartridge or capsule, etc.

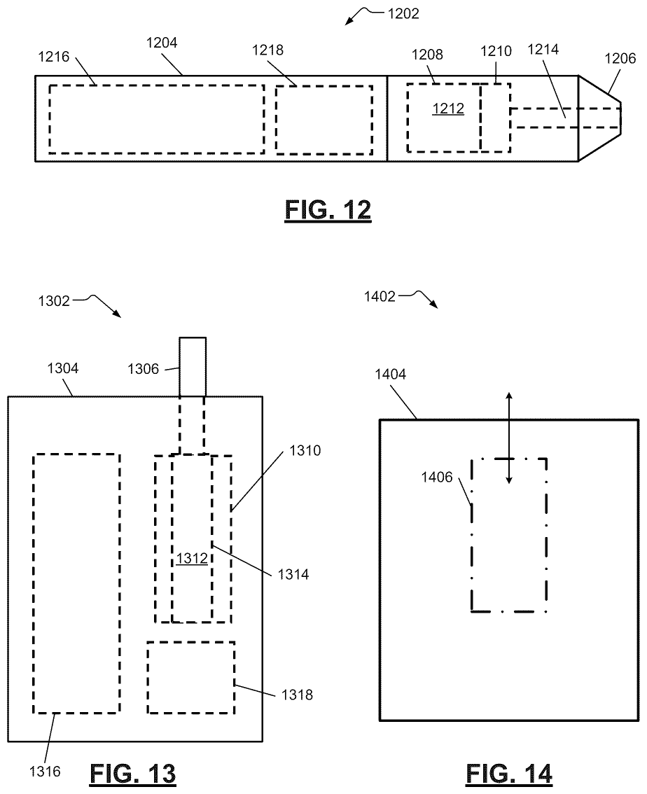

For example, FIGS. 12-14 depict example aerosol-generating devices employable as any one of the aerosol-generating devices. Specifically, in FIG. 12, an e-vaping device 1202 is shown as including a body 1204 and a mouthpiece 1206 integral with, coupled to, or at least partially separable from the body 1204. In the example of FIG. 12, the body 1204 includes a reservoir 1208, a heater 1210 and optionally a ceramic block (not shown). The reservoir 1208 is configured to hold a pre-vapor formulation 1212. The ceramic block is positioned to extend into the reservoir 1208 and a vapor passage 1214 to move the pre-vapor formulation 1212 towards the mouthpiece 1206. The heater 1210 is in thermal contact with the wick and is configured to vaporize the pre-vapor formulation 1212, optionally drawn via the ceramic block, into the vapor passage 1214.

The pre-vapor formulation 1212 is a material or combination of materials that may be transformed into a vapor. For example, the pre-vapor formulation 1212 may be a liquid, solid, and/or gel formulation including, but not limited to, water, beads, solvents, active ingredients, ethanol, plant extracts, natural or artificial flavors, and/or vapor formers such as glycerin and propylene glycol.

The heater 1210 may be configured for resistive heating, inductive heating, infrared heating, ceramic heating, convection heating, microfluidic-channel layered heating, or any combination thereof.

In some example embodiments, the heater 1210 may include or be part of a microfluidics-or chip-based heating assembly. Such a heating assembly may include a plurality of layers. The plurality of layers may be formed from plastic, silicon, titanium, metals, ceramics, polydimethylsiloxane (PDMS), polymers, fiberglass, composites, or other materials. For example, a capillary element of the heating assembly may include a substrate defining a plurality of microchannels. The substrate may include materials such as glass, titanium, aluminum, sapphire, silicon carbide, diamond, ceramics, metals, silicon, and the like. A resistive element of the heating assembly may include a thin film resistive heating element. The thin film resistive heating element may include a low thermal conductivity material, such as, but not limited to, a glass, a plastic, a polymer, a fiberglass, a composite, a ceramic, and the like.

In some example embodiments, the heater 1210 may be a conductive structure that is configured to surround, hold, abut, or otherwise interface with a capillary element containing a pre-vapor formulation (e.g., wire coil surrounding a wick, a conductive mesh abutting an absorbent pad, a metal/alloy trace formed on a ceramic material). The conductive structure may be formed of an electrically resistive material. Examples of suitable electrically resistive materials include titanium, zirconium, tantalum, copper, and/or metals from the platinum group. Examples of suitable metal alloys include stainless steel, nickel-, cobalt-, chromium-, aluminum-titanium-zirconium-, hafnium-, niobium-, molybdenum-, tantalum-, tungsten-, tin-, gallium-, manganese-, and iron-containing alloys, and super-alloys based on nickel, iron, cobalt, stainless steel. For example, the heater may be formed of nickel aluminides (a material with a layer of alumina on the surface), iron aluminides, and other composite materials. The electrically resistive material may optionally be embedded in, encapsulated, or coated with an insulating material or vice-versa, depending on the kinetics of energy transfer and the external physicochemical properties required. In some example embodiments, the heater may include stainless steel, copper alloys, nickel-chromium alloys, iron-chromium alloys, superalloys, and combinations thereof. In some example embodiments, the heater may be a ceramic heater having an electrically resistive layer on an outside surface thereof. In some example embodiments, the heater may be constructed of an iron-aluminide (e.g., FeAl or Fe₃Al), such as those described in U.S. Pat. No. 5,595,706, or nickel aluminides (e.g., Ni3Al), the entire contents of which are hereby incorporate by reference.

As shown in FIG. 12, the body 1204 further includes a power source 1216 and a controller 1218. The power source 1216 is electrically connected to the heater 1210 and is configured to supply an electric current to the heater 1210 during vaping.

In the example of FIG. 12, the power source 1216 may include one or more batteries. In such examples, it should be understood that the shape of the battery (or batteries) for the power source 1216 may vary. For example, the battery may be cylindrical, prismatic, disc-shaped, a pouch battery, or any other variation of battery shape known in the art. Additionally, it should be understood that the battery may be any of a variety of types. For example, in one embodiment, the battery may be a rechargeable battery (e.g., lithium-ion). In another embodiment, the battery may be a non-rechargeable battery (e.g., alkaline). In yet another embodiment, the battery may include silver oxide, carbon zinc, cadmium, nickel, or any another material known in the art. Furthermore, the battery may include a primary cell and/or a secondary cell. It will be understood by those of ordinary skill in the art that various changes in form and details of the battery may be made without departing from the spirit and the scope of the disclosure.

FIG. 13 depicts a heated tobacco device 1302 including a body 1304 and a mouthpiece 1306. In the example of FIG. 13, the aerosol-generating device 1302 is configured to be used with an aerosol forming substrate 1312. The aerosol-generating substrate 1312 may be in a substrate container 1314, as shown, or an aerosol-generating substrate may be configured to be directly received in the body 1304 without a distinct container. In various embodiments, a consumable including the aerosol-forming substrate 1312 and/or a mouthpiece may be employed (e.g., instead of the mouthpiece 1306).

As shown, the device 1302 further includes a heater 1310, a power source 1316, and a controller 1318. In some examples, the heater 1310 may include an external heater, such as a crucible heater. In other examples, the heater 1310 may include a blade that extends into the aerosol-forming substrate 1312 (e.g., at a radial center of the substrate). The heater 1310 may be part of the device 1302, or a consumable including the aerosol-forming substrate 1312. The power source 1316 and the controller 1318 may include a similar arrangement and components as the power source 1216 and the controller 1218 of FIG. 12 explained above.

In various embodiments, a pre-vapor formulation and an aerosol-forming substrate (e.g., the pre-vapor formulation 1212 of FIG. 12, the aerosol-forming substrate 1312 of FIG. 13, etc.) are a material or combination of materials that may yield an aerosol. An aerosol relates to the matter generated or output by the devices disclosed, claimed, and equivalents thereof. The material may include a compound (e.g., nicotine, cannabinoid, cannabimimetic agent, caffeine, etc.) that is released when the material is heated. In such an instance, an aerosol including the compound is produced when the material is heated. The heating may be below the ignition temperature to avoid a self-sustaining burning or a self-sustaining combustion of the material (i.e., in contrast to where a material is lit, such as lit-end cigarettes).

The material(s) may include a fibrous material. For instance, the fibrous material may be a botanical material. The fibrous material is configured to release a compound when heated. The compound may be a naturally occurring constituent of the fibrous material. For instance, the fibrous material may be plant material such as tobacco, and the compound released may be nicotine. The term “tobacco” includes any tobacco plant material including tobacco leaf, tobacco plug, reconstituted tobacco, compressed tobacco, shaped tobacco, or powder tobacco, and combinations thereof from one or more species of tobacco plants, such as Nicotiana rustica and Nicotiana tabacum.

In some example embodiments, the tobacco material may include material from any member of the genus Nicotiana. In addition, the tobacco material may include a blend of two or more different tobacco varieties. The tobacco material may be provided in any suitable form, including, but not limited to, tobacco lamina, processed tobacco materials, such as volume expanded or puffed tobacco, processed tobacco stems, such as cut-rolled or cut-puffed stems, reconstituted tobacco materials, blends thereof, and the like. In some example embodiments, the tobacco material is in the form of a substantially dry tobacco mass. Furthermore, in some instances, the tobacco material may be mixed and/or combined with at least one of propylene glycol, glycerin, sub-combinations thereof, or combinations thereof.

The compound in the generated aerosol may also be a naturally occurring constituent of a medicinal plant that has a medically-accepted physiological effect (e.g., therapeutic effect, prophylactic effect). For instance, the medicinal plant may be a cannabis plant or a cannabimimetic plant (i.e., a plant with similar pharmacological effects to those of cannabis). For a cannabis plant, the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). The fibrous material may include the leaf and/or flower material from one or more species of cannabis plants such as Cannabis sativa, Cannabis indica, and Cannabis ruderalis. In some instances, the fibrous material is a mixture of 60-80% (e.g., 70%) Cannabis sativa and 20-40% (e.g., 30%) Cannabis indica. For a cannabimimetic plant, the compound may be a cannabimimetic agent. Cannabimimetic agents interact with receptors in the body to produce similar pharmacological effects as cannabinoids.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In an example embodiment, heat from a heater (e.g., of the heater 1310 shown in FIG. 13) may cause decarboxylation to convert the tetrahydrocannabinolic acid (THCA) to tetrahydrocannabinol (THC), and/or to convert the cannabidiolic acid (CBDA) to cannabidiol (CBD).

In instances where both tetrahydrocannabinolic acid (THCA) and tetrahydrocannabinol (THC) are present, the decarboxylation and resulting conversion will cause a decrease in tetrahydrocannabinolic acid (THCA) and an increase in tetrahydrocannabinol (THC). At least 50% (e.g., at least 87%) of the tetrahydrocannabinolic acid (THCA) may be converted to tetrahydrocannabinol (THC) during the heating. Similarly, in instances where both cannabidiolic acid (CBDA) and cannabidiol (CBD) are present, the decarboxylation and resulting conversion will cause a decrease in cannabidiolic acid (CBDA) and an increase in cannabidiol (CBD). At least 50% (e.g., at least 87%) of the cannabidiolic acid (CBDA) may be converted to cannabidiol (CBD) during the heating.

Furthermore, the compound which is released may be or may additionally include a non-naturally occurring additive that is subsequently introduced into the fibrous material. In one instance, the fibrous material may include at least one of cotton, polyethylene, polyester, rayon, combinations thereof, or the like (e.g., in a form of a gauze). In another instance, the fibrous material may be a cellulose material (e.g., non-tobacco and/or non-cannabis material). In either instance, the compound introduced may include nicotine, cannabinoids, cannabimimetic agents, and/or flavorants. The flavorants may be from natural sources, such as plant extracts (e.g., tobacco extract, cannabis extract, cannabimimetic extract), and/or artificial sources. In yet another instance, when the fibrous material includes tobacco and/or cannabis, the compound may be or may additionally include one or more flavorants (e.g., menthol, mint, vanilla). Thus, the compound within the aerosol-forming substrate may include naturally occurring constituents and/or non-naturally occurring additives. In this regard, it should be understood that existing levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids. Likewise, the existing levels of one or more cannabimimetic agents in a quantity of cannabimimetic material may be increased through supplementation with an extract containing such cannabimimetic agents.

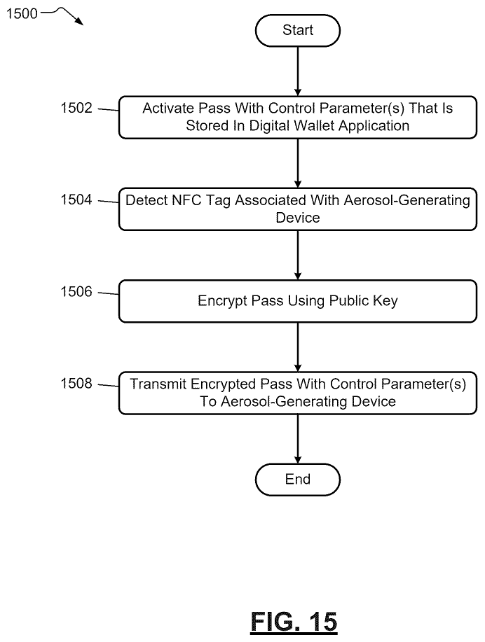

In FIG. 14, an aerosol-generating device 1402 is shown as including a body 1404 configured to receive a consumable 1406 designed to be depleted/expended via use. In such examples, the body 1404 may include or define a recess, a cavity, etc. for receiving the consumable 1406 inserted therein. When desired, the consumable 1406 may be removed from the recess, the cavity, etc. of the aerosol-generating device 1402. In various embodiments, the aerosol-generating device 1402 may include a movable lid (not shown) that can rotate about, separate from, etc. the body 1404. In such examples, the lid can be moved to provide and/or restrict access to the recess, the cavity, etc. and also the consumable 1406 if inserted into the device 1402. In various embodiments, the consumable 1406 may include a heater, an aerosol-forming substrate (e.g., as explained above), a pre-vapor formulation, a mouthpiece, etc. In other examples, one or more of the heater, the aerosol-forming substrate, the pre-vapor formulation, the mouthpiece, etc. may be a part of the aerosol-generating device 1402.

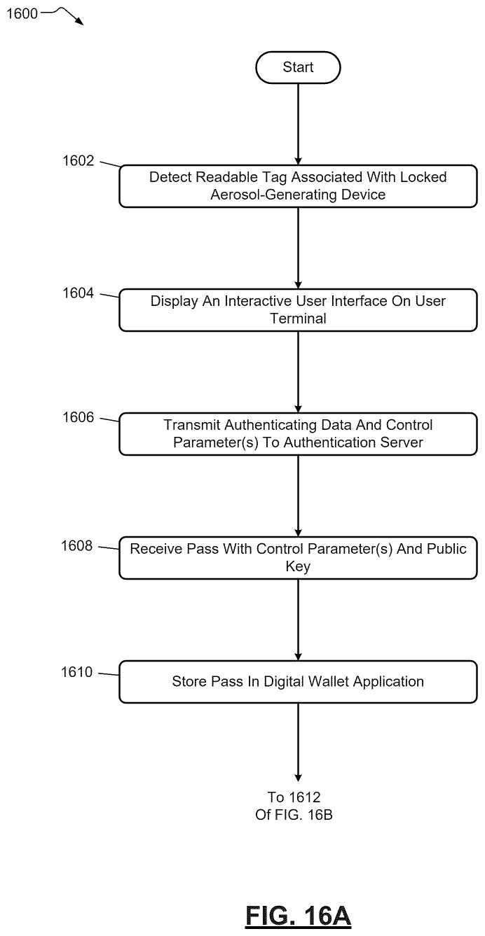

FIGS. 15-16A-B illustrate example control methods 1500, 1600 for unlocking an aerosol-generating device, such as the aerosol-generating device 102 of FIG. 1. In various embodiments, the control methods 1500, 1600 may be implemented at the controller 118 of the operator terminal 104 in FIG. 1. As one example, the control methods 1500, 1600 may be implemented as part of a device manager Finite State Machine (FSM) software implementation executed at the controller 118.

As shown in FIG. 15, the control method 1500 begins at 1502, where the pass 124 stored in the digital wallet application 122 of the operator terminal 104 is activated, as explained above. For example, a consumer may provide user input to activate the pass 124. For instance, and as explained above, the consumer may select the pass 124 in the digital wallet application 122, select an activate input associated with the pass 124, etc. The control method 1500 then proceeds to 1504.

At 1504, the controller 118 detects the NFC tag 114 associated with the aerosol-generating device 102. In such examples, the operator terminal 104 may be placed near or in contact with the aerosol-generating device 102. With this arrangement, the NFC reader 136 of the operator terminal 104 may be placed near the NFC tag 114, enabling the detection of the NFC tag 114 of the device 102. The control method 1500 then proceeds to 1506.

At 1506, the controller 118 encrypts the pass 124 using the public key 126. For example, the controller 118 may cause the digital wallet application 122 to scramble, change, etc. a string data message (e.g., the string data message 450 of FIG. 5) of the pass 124, based on the public key 126. The control method 1500 then proceeds to 1508.

At 1508, the controller 118 causes the operator terminal 104 to transmit the encrypted pass 124 to the aerosol-generating device 102. For example, and as explained above, the controller 118 may enable or activate the NFC tag 134, to allow the NFC reader 116 of the aerosol-generating device 102 to retrieve or otherwise receive the encrypted pass 124. The control method 1500 may end as shown in FIG. 15.

In FIGS. 16A-B, the control method 1600 is similar to the control method 1500 of FIG. 15, but with additional and/or alternative steps. For example, and as shown in FIG. 16A, the control method 1600 begins at 1602, where the controller 118 detects a readable tag associated with the aerosol-generating device 102 in a locked state. In such examples, the readable tag associated with the aerosol-generating device 102 may be an NFC tag (e.g., the NFC tag 114 of the device 102, an NFC tag of the storage case 140, a QR code of the storage case 140, etc.). The control method 1600 then proceeds to 1604.

At 1604, the controller 118 causes display of an interactive user interface on the operator terminal 104. For example, and as explained above, in response to detecting the readable tag associated with the aerosol-generating device 102, the controller 118 may control the display 138 to automatically render the interactive user interface via a website, present a link to the website with the interactive user interface, etc. The control method 1600 then proceeds to 1606.

At 1606, the controller 118 transmits authenticating data and control parameter(s) to the authentication server 106. For example, and as explained above, a consumer may use the interactive user interface to upload a picture of an identification (e.g., a driver's license, a passport, etc.), input a date of birth, and/or specific credentials of the consumer. Also, during this time, the consumer may select other inputs, such as a specific operating system and/or device brand of the operator terminal 104, an owner of the device 102, one or control parameter(s) for the device 102, etc. Then, once the necessary data is provided, the consumer may select an input to case the controller 118 to transmit the provided data to the authentication server 106. The control method 1600 then proceeds to 1608.

At 1608, the controller 118 receives the created pass 124 provided by the authentication server 106 and the public key 126. In this example, the created pass 124 includes the control parameter(s) for the device 102. For example, and as explained above, the authenticating data provided by the consumer appropriately verifies the consumer, including his/her age, the authentication server 106 may create the pass 124 as explained above. Once created, the controller 118 may cause the display 138 to present a link to download the pass 124 and the public key 126 from the authentication server 106. The control method 1600 then proceeds to 1610.

At 1610, the controller 118 stores the pass 124 in the digital wallet application 122. The control method 1600 then proceeds to 1612 of FIG. 16B.

At 1612, the controller 118 determines whether the pass 124 is activated for use. For example, the pass 124 may be required to be activated before use. In such examples, the pass 124 may be activated in response to user input as explained above. For instance, the consumer may be required to take active steps to activate the pass 124 by selecting the pass 124. In other examples, the aerosol-generating device 102 may detect and prompt the operator terminal 104 to notify the consumer that the device 102 is nearby. In such examples, the consumer may then provide input to activate the pass 124. If the pass 124 is determined to be activated, the control method 1600 proceeds to 1614. If not, the control method 1600 may return to 1612.

At 1614, the controller 118 determines whether an NFC tag is detected, such as the NFC tag 114 of the aerosol-generating device 102. For example, the operator terminal 104 may be placed near or in contact with the aerosol-generating device 102. In such examples, the NFC reader 136 of the operator terminal 104 may be placed near the NFC tag 114. With this arrangement, the NFC reader 136 may detect the NFC tag 114 of the device 102. If the NFC tag is detected, the control method 1600 proceeds to 1506, 1508 of FIG. 15 explained above, and then may end.

Reverting back to 1614, if the NFC tag is not detected, the control method 1600 proceeds to 1620. At 1620, the controller 118 determines whether a defined period of time (e.g., a time out period) has elapsed. If yes, the controller 118 deactivates the pass 124 at 1622. Then, the control method 1600 returns to 1612. Otherwise, if no at 1620, the control method 1600 returns to 1614.

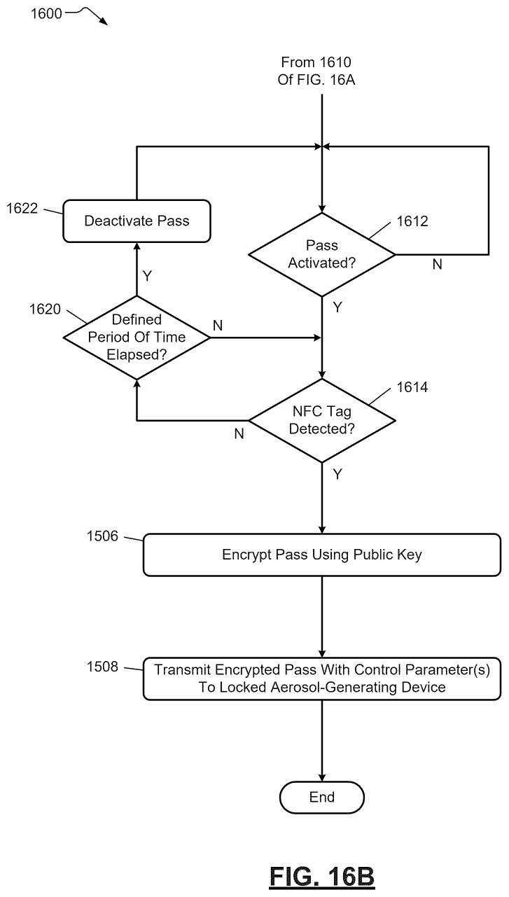

FIGS. 17-19 illustrate example control methods 1700, 1800, 1900 for unlocking an aerosol-generating device, such as the aerosol-generating device 102 of FIG. 1. In various embodiments, the control methods 1700, 1800, 1900 may be implemented at the controller 108 of the aerosol-generating device 102 in FIG. 1. As one example, the control methods 1700, 1800, 1900 may be implemented as part of a device manager Finite State Machine (FSM) software implementation executed at the controller 108.

As shown in FIG. 17, the control method 1700 begins at 1702, where the controller 108 receives the encrypted pass 124 from, for example, the operator terminal 104. In this example, the encrypted pass 124 includes one or more control parameters for the aerosol-generating device 102, as explained above. The control method 1700 then proceeds to 1704.

At 1704, the controller 108 decrypts the received pass 124. For example, and as explained above, the controller 108 may retrieve the private key 112 from the memory 110 in the aerosol-generating device 102, and then decrypt the received pass 124 (e.g., the string data message 450) using the private key 112. The control method 1700 then proceeds to 1706.

At 1706, the controller 108 unlocks the aerosol-generating device 102. For example, to unlock the device 102, the controller 108 may connect a power source and a heater in the device 102, clear a locking flag or bit set in the memory 110, set an unlock flag or bit in the memory 110, etc. The control method 1700 then proceeds to 1708.

At 1708, the controller 108 controls the aerosol-generating device 102 based on the control parameter(s) in the pass 124. For example, the controller 108 may control the device 102 based on an unlock interval setting, a temperature setting, a puff profile, a power setting, an interface setting, a consumer notification setting, and/or another suitable control parameter provided with the pass 124. The control method 1700 may end as shown in FIG. 17.

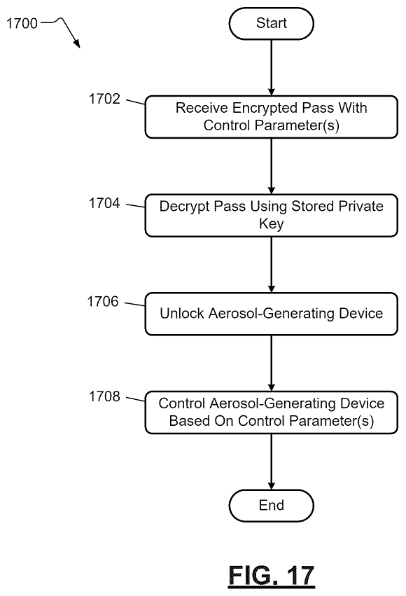

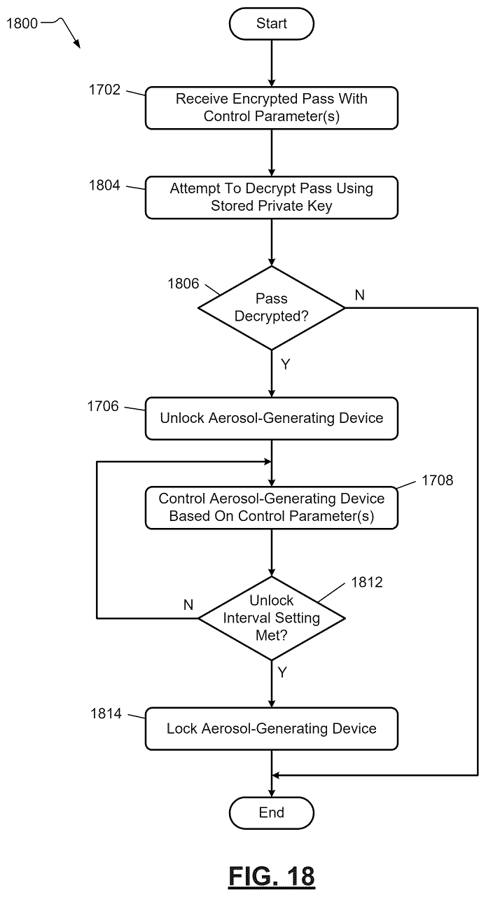

In FIG. 18, the control method 1800 is similar to the control method 1700 of FIG. 17, but with additional and/or alternative steps. For example, and as shown in FIG. 18, the control method 1800 begins at 1702 of FIG. 17 explained above, and then proceeds to 1804. At 1804, the controller 108 attempts to decrypt the received pass 124. For example, and as explained above, the controller 108 may retrieve the private key 112 from the memory 110 in the aerosol-generating device 102, and then attempt to decrypt the received pass 124 (e.g., the string data message 450) using the private key 112. The control method 1800 then proceeds to 1806.

At 1806, the controller 108 determines whether the pass 124 was decrypted. For example, if the pass 124 is decrypted, a flag or bit may be set. In such examples, the controller 108 may read the flag or bit. If yes, the control method 1800 then proceeds to 1706, 1708 of FIG. 17 explained above, and to 1812. Otherwise, if no at 1806 indicating the pass 124 is invalid, the control method 1800 may end as shown in FIG. 18.

At 1812, the controller 108 determines whether the unlock interval setting is met. For example, the unlock interval setting may specify a condition for transitioning back to a locked state, such as a defined period of time, a defined number of puffs, an amount consumed, etc. In such examples, the controller 108 may determine whether the condition is met. If yes, the control method 1800 proceeds to 1814. Otherwise, if no at 1812, the control method 1800 returns to 1708.

At 1814, the controller 108 locks the aerosol-generating device 102. For example, to lock the device 102, the controller 108 may disconnect the power source in the device 102 from the heater, set a locking flag or bit, etc. The control method 1800 may then end.

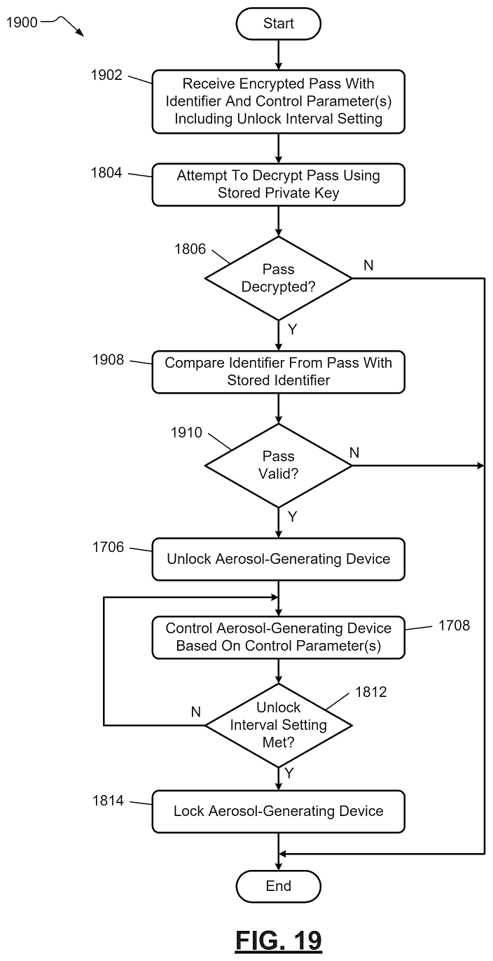

In FIG. 19, the control method 1900 is similar to the control methods 1700, 1800 of FIGS. 17-18, but with additional and/or alternative steps. For example, and as shown in FIG. 19, the control method 1900 begins at 1902, where the controller 108 receives the encrypted pass 124 from, for example, the operator terminal 104. In this example, the encrypted pass 124 includes an identifier and one or more control parameters for the aerosol-generating device 102, as explained above. The control method 1900 then proceeds to 1804, 1806 of FIG. 18 explained above, and to 1908.

At 1908, the controller 108 compares the retrieved identifier from the pass 124 with a device identifier stored in the memory 110 of the device 102. In such examples, the identifier on the device 102 may be set before, during, and/or after manufacturing of the device 102. The control method 1900 then proceeds to 1910.

At 1910, the controller 108 determines whether the received pass 124 (e.g., the received string data message 450) is valid based on the comparison. For instance, if the retrieved identifier from the pass 124 and the stored device identifier match, the pass 124 may be valid. If yes, the control method 1900 proceeds to 1706, 1708 of FIG. 17 explained above, then to 1812, 1814 of FIG. 18 explained above, and then may end. Otherwise, if no at 1910, the control method 1900 may end.

FIG. 20 illustrates an example control method 2000 for verifying a consumer and generating a pass for unlocking an aerosol-generating device, such as the aerosol-generating device 102 of FIG. 1. In various embodiments, the control method 2000 may be implemented at the controller 128 of the authentication server 106 in FIG. 1. As one example, the control method 2000 may be implemented as part of a device manager Finite State Machine (FSM) software implementation executed at the controller 128.

As shown in FIG. 20, the control method 2000 begins at 2002, where the controller 128 receives authenticating data and control parameter(s) from the operator terminal 104. For example, and as explained above, a consumer may use an interactive user interface displayed by the operator terminal 104 to upload a picture of an identification (e.g., a driver's license, a passport, etc.), input a date of birth, and/or specific credentials of the consumer. Additionally, the consumer may select or otherwise provide other inputs, such as a specific operating system and/or device brand of the operator terminal 104, an owner of the device 102, an identifier (e.g., a device ID, a SKU, etc.) associated with the device 102 one or control parameter(s) for the device 102, etc. This data may then be provided to the authentication server 106. The control method 2000 then proceeds to 2004.

At 2004, the controller 128 retrieves data from the database 132. For example, the controller 128 may send a request to the database 132 seeking data pertaining to the consumer. In such examples, the database 132 may be a secured database (e.g., a third-party database) accessible by the authentication server 106. The control method 2000 then proceeds to 2006.

At 2006, the controller 128 compares the authenticating data from the operator terminal 104 and the consumer data from the database 132. More specifically, the controller 128 may compare the provided consumer credentials from the consumer with the verified consumer data from the database 132. The control method 2000 then proceeds to 2008.

At 2008, the controller 128 determines whether the consumer is authenticated (e.g., age verified) based on the authenticating data and the consumer data. If yes, the control method 2000 proceeds to 2010.

At 2010, the controller 128 receives device data. For example, at 1610, the controller 128 may receive the device identifier from the operator terminal 104 at 1602. In other examples, the controller 128 may retrieve a device identifier from the database 132 or another suitable database based on one or more of the device related inputs (e.g., the device ID, the SKU, etc.) received at 1602. The control method 2000 then proceeds to 2012.

At 2012, the controller 128 determines whether the received device identifier is verified. For example, the controller 128 may determine that the device identifier is verified if the device identifier is present in a database (e.g., the database 132 or another suitable database), has not been used before, corresponds to the device ID, the SKU, etc. If the received device identifier is verified (yes at 2012), the control method 2000 proceeds to 2018 explained below. Otherwise, if the received device identifier is not verified (no at 2012), the control method 2000 proceeds to 2016.

At 2014, the controller 128 generates the pass 124 specific to the verified consumer. In such examples, the pass 124 may include a string data message including, for example, the received control parameter(s), an identifier for the device 102, a SKU, etc. The control method 2000 then proceeds to 2016.

At 2016, the controller 128 transmits the pass 124 with the control parameter(s) and possibly other information, and a public key associated with the pass 124 to the operator terminal 104. As one example, the controller 128 may push a link to the operator terminal 104 for display. This link may be selected to download the pass 124 and the public key from the authentication server 106. The control method 2000 may then end.

Reverting back to 2008, if the consumer is not authenticated, the control method 2000 proceeds to 2018. At 2018, the controller 128 may generate and transmit a notification indicating a failed authentication, an unverified device identifier, etc. to, for example, the operator terminal 104. The control method 2000 may then end.

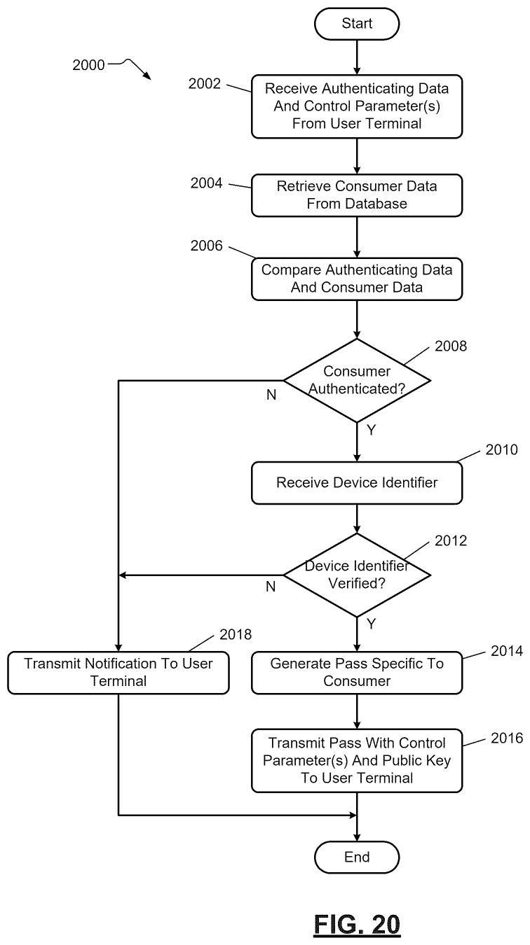

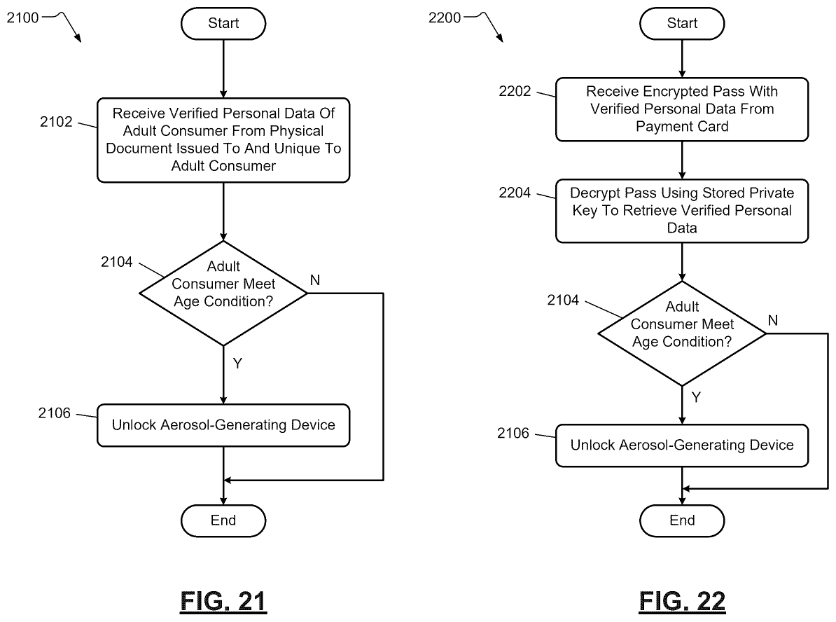

FIGS. 21-24 illustrate example control methods 2100, 2200, 2300, 2400 for unlocking an aerosol-generating device, such as the aerosol-generating device 602 of FIGS. 6 and/or 8-9. In various embodiments, the control methods 2100, 2200, 2300, 2400 may be implemented at the controller 108 of the aerosol-generating device 602 in FIGS. 6 and/or 8-9. As one example, the control methods 2100, 2200, 2300, 2400 may be implemented as part of a device manager Finite State Machine (FSM) software implementation executed at the controller 108.

As shown in FIG. 21, the control method 2100 begins at 2102, where the controller 108 of the aerosol-generating device 602 receives verified personal data of an adult consumer from a physical document issued to and unique to that adult consumer. In this example, the verified personal data (or a portion or representative thereof) is received from a physical document, such as a government-issued biometric ID (e.g., a biometric passport, a biometric driver's license, etc.) containing verified customer data, a payment card (e.g., a bank card, etc.) containing verified customer data, etc. In such examples, the controller 108 receives the verified personal data via a wireless communication, such as NFC. For example, the verified personal data may be received by the aerosol-generating device 602 when the physical document is placed near or in contact with the aerosol-generating device 602, thereby enabling the NFC reader 116 of the aerosol-generating device 602 to detect a wireless tag (e.g., an NFC tag) of the physical document and initiate receipt of the verified personal data. The control method 2100 then proceeds to 2104.

At 2104, the controller 108 determines whether the adult consumer meets an age condition for unlocking the aerosol-generating device 602. For example, the controller 108 can determine whether the adult consumer meets an age condition based on the verified personal data received from the physical document. In such examples, based on the verified personal data, the controller 108 can receive a bit value signifying the adult consumer is verified for using the aerosol-generating device 602 (e.g., of an appropriate age), determine whether the age of the adult consumer is greater than a threshold, etc. If yes at 2104, the control method 2100 proceeds to 2106. Otherwise, if no at 2104, the control method 2100 may end.

At 2106, the controller 108 unlocks the aerosol-generating device 602. In such examples, to unlock the aerosol-generating device 602, the controller 108 may connect a power source (e.g., a battery, etc.) and a heater in the device 602 by, for example, generating a control signal to close a switch between the components. In other examples, the controller 108 may clear a locking flag or bit set in the memory 110 of the aerosol-generating device 602, set a flag or bit in the memory 110, etc. The control method 2100 may then end.

In FIG. 22, the control method 2200 begins at 2202, where the controller 108 of the aerosol-generating device 602 receives an encrypted pass with verified personal data of an adult consumer from a payment card issued to and unique to that adult consumer. The payment card may be a debit card, a credit card, or any other suitable contactless bank card issued by a financial institution to the adult consumer following a verification process to authenticate the consumer. In such examples, the pass can be created specifically for the authenticated adult consumer by an operator terminal (e.g., the operator terminal 604 of FIG. 8) and/or another source (e.g., the authentication server 106 of FIG. 1 and/or another suitable consumer authenticating device) and then provided to the operator terminal. With this approach, the pass may include the verified personal data obtained from, for example, during an authenticating process, a mobile identification, etc. Then, the pass can be encrypted with a public key at the operator terminal and passed to the payment card, where the payment can store the encrypted pass. In some examples, the encrypted pass may include the verified personal data along with other data if desired, such as a unique identifier of the aerosol-generating device 602, control parameter(s) for the aerosol-generating device 602, etc. as explained above. The control method 2200 then proceeds to 2204.

At 2204, the controller 108 decrypts the pass with a private key stored on the aerosol-generating device 602. Once decrypted, the controller 108 can retrieve the verified personal data from the pass, along with other data contained therein (if applicable). The control method 2200 then proceeds to 2104 of FIG. 21 explained above. Then, if yes at 2104, the control method 2200 proceeds to 2106 of FIG. 21 explained above. Otherwise, if no at 2104, the control method 2100 may end.

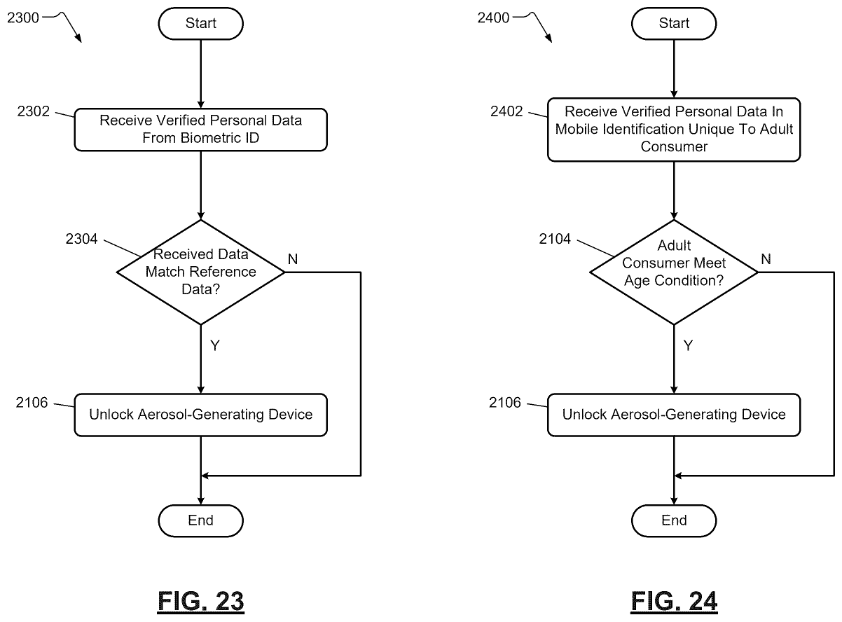

In FIG. 23, the control method 2300 begins at 2302, where the controller 108 of the aerosol-generating device 602 receives verified personal data of an adult consumer from a biometric ID issued to and unique to that adult consumer. The biometric ID may be a biometric passport, a biometric driver's license, etc. issued by a government institution to the adult consumer following a verification process to authenticate the consumer. In such examples, the verified personal data may include one or more pieces of information for identifying and verifying the adult consumer, such as identification information unique to the adult consumer (e.g., a name, a date of birth, etc.), biometric information (e.g., a facial image, a fingerprint, etc.), etc. of the adult consumer. The controller 108 receives the verified personal data from the biometric ID via a wireless communication, such as NFC. For example, the verified personal data may be received by the aerosol-generating device 602 when the biometric ID is placed near or in contact with the aerosol-generating device 602, thereby enabling the NFC reader 116 of the aerosol-generating device 602 to detect an NFC tag of the biometric ID and initiate receipt of the verified personal data.

Then, the controller 108 determines whether the adult consumer meets an age condition for unlocking the aerosol-generating device 602 based on the verified personal data. More specifically, at 2304, the controller 108 can determine whether the received data matches reference data stored, for example, in the memory 110 of the aerosol-generating device 602. For instance, a biometric of the adult consumer meeting the age condition may be stored in the memory 110 and used as a reference (e.g., reference data 912 in FIG. 9) to compare with biometric information provided by the biometric ID. In such examples, the biometric of the adult consumer (e.g., a facial image, a fingerprint, etc.) may be established with an external device (e.g., the operator terminal 604 of FIG. 6) and provided to the device 602 or established with a biometric sensor (e.g., a fingerprint sensor, etc.) on the device 602 itself. In other examples, identification information of the adult consumer meeting the age condition may be stored in the memory 110 and used as a reference (e.g., reference data 912 in FIG. 9) to compare with identification information provided by the biometric ID. With this approach, the adult consumer may provide the reference identification information during an authentication process, as explained above.

If the controller 108 determines the adult consumer meets the age condition (e.g., the received data matches the stored reference data) at 2304 (yes at 2304), the control method 2300 proceeds to 2106 of FIG. 21 explained above. Otherwise, if no at 2304, the control method 2300 may end.

In FIG. 24, the control method 2400 begins at 2402, where the controller 108 of the aerosol-generating device 602 receives verified personal data contained in a mobile identification (e.g., the mobile identification 618 of FIG. 6) and unique to an adult consumer. In such examples, the mobile identification containing the verified personal data may be stored in the digital wallet application 622 of the operator terminal 604. The mobile identification may be retrieved from a government-controlled source, such as through a government-regulated website, a government-regulated application, etc., as explained above. In other examples, the controller 108 of the aerosol-generating device 602 may alternatively receive the verified personal data contained in a proprietary ID stored in the digital wallet application 622 of the operator terminal 604 and retrieved from a private source capable of identity and age verification, as explained above.

Additionally, in some examples, the verified personal data (in the mobile identification or the proprietary ID) received from the digital wallet application 622 of the operator terminal 604 may be contained in an encrypted pass, as explained above. In such examples, the pass can be encrypted with a public key at the operator terminal 604 and then passed to the controller 108. Once received, the controller 108 can decrypt the pass with a private key stored on the aerosol-generating device 602 and retrieve the verified personal data from the pass, along with other data (e.g., control parameter(s) for the aerosol-generating device 602) contained therein if applicable.

Then, after the verified personal data is received at 2402, the control method 2400 proceeds to 2104 of FIG. 21 explained above. Then, if yes at 2104, the control method 2400 proceeds to 2106 of FIG. 21 explained above. Otherwise, if no at 2104, the control method 2400 may end.

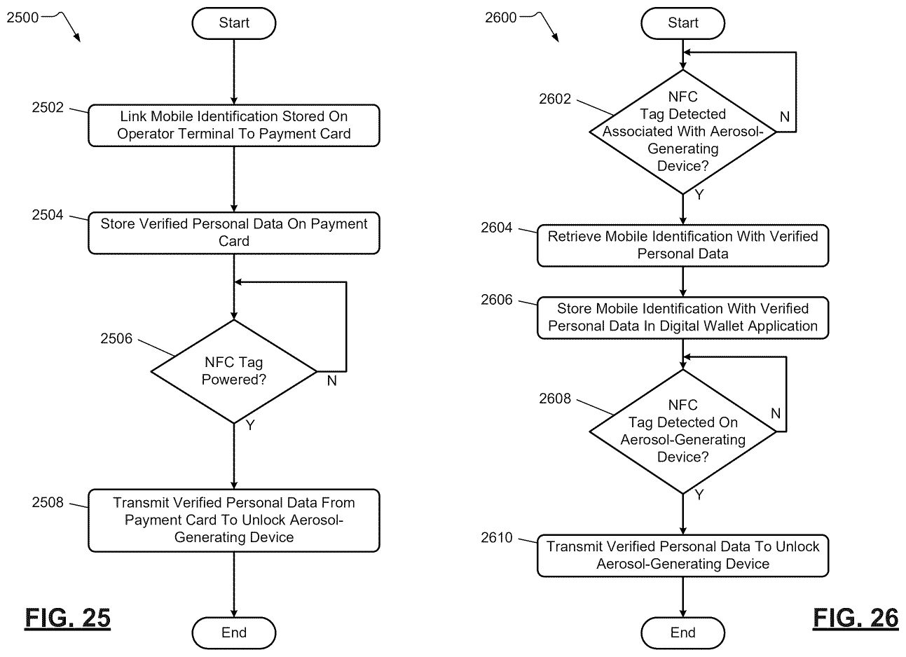

FIGS. 25-26 illustrate example control methods 2500, 2600 for transmitting verified personal data for unlocking an aerosol-generating device, such as the aerosol-generating device 602 of FIGS. 6 and/or 8. In various embodiments, some or all steps of the control methods 2500, 2600 may be implemented at the controller 118 of the of the operator terminal 604 in FIGS. 6 and/or 8. As one example, the control methods 2500, 2600 may be implemented as part of a device manager Finite State Machine (FSM) software implementation executed at the controller 118.

As shown in FIG. 25, the control method 2500 begins at 2502, where the controller 118 of the operator terminal 604 links a mobile identification, such as the mobile identification 618 of FIG. 6 stored on the operator terminal 604 with a payment card, such as the payment card 808 of FIG. 8. For example, the controller 118 may provide a selectable input on the operator terminal 604 allowing the adult consumer to link the payment card 808 and the mobile identification 618. In some examples, a digital representation of the payment card 808 and the mobile identification 618 may be stored in the digital wallet application 622. In this example, the adult consumer may access the selectable input through the application 620 on the operator terminal 604 or through the digital wallet application 622. The control method 2500 then proceeds to 2504.

At 2504, the controller 118 stores verified personal data on the payment card 808. For example, the operator terminal 604 can wirelessly communicate the verified personal data contained in the mobile identification 618 to the payment card 808, which then stores the data (e.g., the verified personal data 828) in the memory 826. In such examples, the operator terminal 604 and the payment card 808 can communicate via NFC through the NFC reader 136 of the operator terminal 604 and the NFC tag 830 of the payment card 808, as explained above. In some examples, the verified personal data received from the operator terminal 604 may be contained in an encrypted pass, as explained above. The control method 2500 then proceeds to 2506.

At 2506, a determination is made as to whether the NFC tag 830 in the payment card 808 is powered. For example, when the payment card 808 is placed in the vicinity of the NFC reader 116 of the aerosol-generating device 602, the payment card 808 receives a signal from the NFC reader 116 that powers the NFC tag 830 in the card 808. If yes at 2506, the control method 2500 proceeds to 2508. Otherwise, if no at 2506, the control method 2500 may return to 2506.

At 2508, the verified personal data stored on the payment card 808 is transmitted from the payment card 808 to the aerosol-generating device 602 for unlocking the device 602. If the verified personal data is contained in an encrypted pass, the pass may be transmitted from the payment card 808 to the aerosol-generating device 602. Regardless of whether the verified personal data is in an encrypted pass, the verified personal data can be transmitted via the powered NFC tag 830 in the payment card 808 and the NFC reader 136 of the operator terminal 604, as explained herein. The control method 2500 may then end.

In FIG. 26, the control method 2600 begins at 2602, where the controller 118 of the operator terminal 604 determines whether an NFC tag associated with the aerosol-generating device 602 is detected. For example, the operator terminal 604 may be placed near a storage case (e.g., the storage case 140 of FIG. 1) for the aerosol-generating device 602. In this example, the storage case may include an NFC tag readable by the NFC reader 136 of the operator terminal 604. In other examples, the operator terminal 604 may be placed near the aerosol-generating device 602 so that the NFC reader 136 of the operator terminal 604 can read the NFC tag 114 of the device 602. If no at 2602, the control method 2600 returns to 2602. Otherwise, if yes at 2602, the control method 2600 proceeds to 2604.

At 2604, the controller 118 retrieves a mobile identification, such as the mobile identification 618 of FIG. 6 unique to the adult consumer. For example, in response to detecting the NFC tag associated with the aerosol-generating device 602, the operator terminal 604 can be used to initiate one or more events to retrieve the mobile identification 618 from the government-controlled source. For instance, and as explained above, the controller 118 may cause the operator terminal 604 to launch a web browser at a government-regulated website or launch a government-regulated application installed on the operator terminal 604 to communicate with the web server 606 and access the mobile identification 618 through the web server 606. In such examples, the controller 118 may generate a command to launch the web browser or the government-regulated application having an interactive user interface, thereby allowing the adult consumer to input credentials, such as consumer authenticating data as explained above. Once the adult consumer is authenticated, the controller 118 can receive the mobile identification 618 containing verified personal data from the web server 606. In some examples, the verified personal data may be contained in an encrypted pass, as explained above. The control method 2600 then proceeds to 2606.

At 2606, the controller 118 stores the mobile identification 618 containing the verified personal data in the digital wallet application 622 of the operator terminal 604. The control method 2600 then proceeds to 2608.

At 2608, the controller 118 determines whether the NFC tag 114 of the aerosol-generating device 602 is detected. If no, the control method 2600 returns to 2608. Otherwise, if yes at 2608, the control method 2600 proceeds to 2610. For example, the operator terminal 604 and the aerosol-generating device 602 may be placed near each other so that the NFC reader 136 of the operator terminal 604 can read and detect the NFC tag 114 of the device 602. If no at 2602, the control method 2600 returns to 2608. Otherwise, if yes at 2608, the control method 2600 proceeds to 2610.

At 2610, the verified personal data contained in the mobile identification 618 is transmitted to the aerosol-generating device 602 for unlocking the device 602. If the verified personal data is contained in an encrypted pass, the pass may be transmitted to the aerosol-generating device 602. Regardless of whether the verified personal data is in an encrypted pass, the verified personal data can be transmitted from the operator terminal 604 to the aerosol-generating device 602 via NFC, as explained herein. The control method 2600 may then end.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Although described with reference to specific examples and drawings, modifications, additions and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

Illustrative Embodiment 1. A system for unlocking an aerosol-generating device, comprises a physical document issued and unique to an adult consumer, the physical document including at least one first wireless communication chip and memory configured to store verified personal data of the adult consumer, and an aerosol-generating device including at least one second wireless communication chip and a controller. The controller is configured to receive, from the physical document via the first wireless communication chip and the second wireless communication chip, the verified personal data of the adult consumer, determine whether the adult consumer meets an age condition based on the verified personal data, and unlock the aerosol-generating device in response to the adult consumer meeting the age condition.

Illustrative Embodiment 2. The system of illustrative embodiment 1, wherein the physical document is a government-issued identification.

Illustrative Embodiment 3. The system of illustrative embodiment 2, wherein the government-issued identification is one of a biometric passport or a biometric driver's license.

Illustrative Embodiment 4. The system of illustrative embodiment 1, wherein the physical document is a payment card.

Illustrative Embodiment 5. The system of illustrative embodiment 4, wherein the payment card is a contactless bank card.

Illustrative Embodiment 6. The system of illustrative embodiment 4, wherein the memory of the payment card is configured to store an encrypted pass with the verified personal data of the adult consumer, the aerosol-generating device includes memory configured to store a private key, and the controller of the aerosol-generating device is configured to receive the encrypted pass from the payment card and decrypt the encrypted pass using the private key to retrieve the verified personal data of the adult consumer.

Illustrative Embodiment 7. The system of illustrative embodiment 6, wherein the encrypted pass is generic to a plurality of aerosol-generating devices, and wherein the plurality of aerosol-generating devices includes the aerosol-generating device.

Illustrative Embodiment 8. The system of illustrative embodiment 1, wherein the verified personal data of the adult consumer enables activation of at least one active ingredient in the aerosol-generating device.

Illustrative Embodiment 9. The system of illustrative embodiment 8, wherein the at least one active ingredient includes at least one of nicotine, caffeine, and a cannabinoid.

Illustrative Embodiment 10. The system of illustrative embodiment 1, wherein the aerosol-generating device includes an aerosol-forming substrate, a heater configured to heat the aerosol-forming substrate, and a power source configured to provide power to the heater.

Illustrative Embodiment 11. The system of illustrative embodiment 1, wherein the aerosol-generating device includes a pre-vapor formulation, a heater configured to vaporize the pre-vapor formulation, and a power source configured to provide power to the heater.

Illustrative Embodiment 12. The system of illustrative embodiment 1, wherein the second wireless communication chip of the aerosol-generating device includes a wireless reader, and the first wireless communication chip of the physical document includes a wireless tag detectable by the wireless reader of the aerosol-generating device to initiate reception of the verified personal data.

Illustrative Embodiment 13. A method for unlocking an aerosol-generating device, comprises receiving, from a wireless communication chip of a physical document issued and unique to an adult consumer, verified personal data of the adult consumer, determining whether the adult consumer meets an age condition based on the verified personal data, and unlocking the aerosol-generating device in response to the adult consumer meeting the age condition.

Illustrative Embodiment 14. The method of illustrative embodiment 13, wherein the physical document is a government-issued identification.

Illustrative Embodiment 15. The method of illustrative embodiment 14, wherein the government-issued identification is one of a biometric passport or a biometric driver's license.

Illustrative Embodiment 16. The method of illustrative embodiment 13, wherein the physical document is a payment card.

Illustrative Embodiment 17. The method of illustrative embodiment 16, wherein the payment card is a contactless bank card.

Illustrative Embodiment 18. The method of illustrative embodiment 16, wherein the payment card includes memory configured to store an encrypted pass with the verified personal data of the adult consumer, receiving verified personal data of the adult consumer includes receiving the encrypted pass from the payment card, and determining whether the adult consumer meets an age condition based on the verified personal data includes decrypting the encrypted pass using a private key stored on the aerosol-generating device to retrieve the verified personal data of the adult consumer.

Illustrative Embodiment 19. The method of illustrative embodiment 13, further comprising activating at least one active ingredient in the aerosol-generating device based on the verified personal data.

Illustrative Embodiment 20. The method of illustrative embodiment 19, wherein the at least one active ingredient includes at least one of nicotine, caffeine, and a cannabinoid.

Illustrative Embodiment 21. The method of illustrative embodiment 13, wherein the aerosol-generating device includes a wireless reader, the wireless communication chip of the physical document includes a wireless tag, and the method further comprises detecting, by the wireless reader, the wireless tag to initiate reception of the verified personal data.

Illustrative Embodiment 22. A system for unlocking an aerosol-generating device, comprises an operator terminal including a digital wallet application configured to store a mobile identification containing verified personal data unique to an adult consumer, and an aerosol-generating device including a controller configured to receive, from the operator terminal via a wireless communication, the verified personal data in the mobile identification, determine whether the adult consumer meets an age condition based on the verified personal data, and unlock the aerosol-generating device in response to the adult consumer meeting the age condition.

Illustrative Embodiment 23. The system of illustrative embodiment 22, wherein the operator terminal is configured to retrieve the mobile identification unique to the adult consumer from an external web server.

Illustrative Embodiment 24. The system of illustrative embodiment 22, wherein the operator terminal includes a wireless reader and a controller configured to retrieve the mobile identification in response to the wireless reader detecting a wireless tag associated with the aerosol-generating device.

Illustrative Embodiment 25. The system of illustrative embodiment 24, wherein the aerosol-generating device includes the wireless tag.

Illustrative Embodiment 26. The system of illustrative embodiment 24, wherein the wireless tag is included in a storage case for the aerosol-generating device.

Illustrative Embodiment 27. The system of illustrative embodiment 24, wherein the controller of the operator terminal is configured to launch a web browser, installed on the operator terminal, at a government-regulated website where the mobile identification is accessible.

Illustrative Embodiment 28. The system of illustrative embodiment 24, wherein the controller of the operator terminal is configured to launch a government-regulated application where the mobile identification is accessible.

Illustrative Embodiment 29. The system of illustrative embodiment 22, wherein the operator terminal includes a controller configured to initiate a wireless pairing of the operator terminal and the aerosol-generating device.

Illustrative Embodiment 30. The system of illustrative embodiment 29, wherein the controller of the aerosol-generating device is configured to receive the verified personal data in the mobile identification via the wireless pairing of the operator terminal and the aerosol-generating device.

Illustrative Embodiment 31. The system of illustrative embodiment 30, wherein the wireless pairing is a Bluetooth® wireless pairing.

Illustrative Embodiment 32. The system of illustrative embodiment 22, wherein the operator terminal includes a wireless reader and a controller configured to launch the digital wallet application for initiating transmission of the verified personal data in the mobile identification to the aerosol-generating device in response to the wireless reader detecting a wireless tag associated with the aerosol-generating device.

Illustrative Embodiment 33. The system of illustrative embodiment 32, wherein the controller of the aerosol-generating device is configured to receive the verified personal data in the mobile identification via near-field communication (NFC).

Illustrative Embodiment 34. The system of illustrative embodiment 32, wherein the aerosol-generating device includes the wireless tag.

Illustrative Embodiment 35. The system of illustrative embodiment 32, wherein the wireless tag is included in a storage case for the aerosol-generating device.

Illustrative Embodiment 36. A method for unlocking an aerosol-generating device with a mobile identification unique to an adult consumer and stored on an operator terminal, comprises receiving, via a wireless communication, verified personal data in the mobile identification unique to the adult consumer and stored in a digital wallet application of the operator terminal, determining whether the adult consumer meets an age condition based on the verified personal data, and unlocking the aerosol-generating device in response to the adult consumer meeting the age condition.

Illustrative Embodiment 37. The method of illustrative embodiment 36, wherein receiving the verified personal data in the mobile identification includes retrieving the mobile identification unique to the adult consumer from an external web server.

Illustrative Embodiment 38. The method of illustrative embodiment 36, wherein receiving the verified personal data in the mobile identification includes retrieving the mobile identification in response to a wireless tag associated with the aerosol-generating device being detected by the operator terminal.

Illustrative Embodiment 39. The method of illustrative embodiment 38, wherein the aerosol-generating device includes the wireless tag.

Illustrative Embodiment 40. The method of illustrative embodiment 38, wherein the wireless tag is included in a storage case for the aerosol-generating device.

Illustrative Embodiment 41. The method of illustrative embodiment 36, wherein receiving the verified personal data in the mobile identification includes receiving the verified personal data in the mobile identification via a Bluetooth® wireless pairing of the aerosol-generating device and the operator terminal.

Illustrative Embodiment 42. The method of illustrative embodiment 36, wherein receiving the verified personal data in the mobile identification includes receiving the verified personal data in the mobile identification via near-field communication (NFC).

1. A system for unlocking an aerosol-generating device, the system comprising:
a physical document issued and unique to an adult consumer, the physical document including at least one first wireless communication chip and memory configured to store verified personal data of the adult consumer; and
an aerosol-generating device including at least one second wireless communication chip and a controller configured to,
receive, from the physical document via the first wireless communication chip and the second wireless communication chip, the verified personal data of the adult consumer;
determine whether the adult consumer meets an age condition based on the verified personal data; and
unlock the aerosol-generating device in response to the adult consumer meeting the age condition.

2. The system of claim 1, wherein the physical document is a government-issued identification.

3. The system of claim 2, wherein the government-issued identification is one of a biometric passport or a biometric driver's license.

4. The system of claim 1, wherein the physical document is a payment card.

5. The system of claim 4, wherein the payment card is a contactless bank card.

6. The system of claim 4, wherein:
the memory of the payment card is configured to store an encrypted pass with the verified personal data of the adult consumer;
the aerosol-generating device includes memory configured to store a private key; and
the controller of the aerosol-generating device is configured to receive the encrypted pass from the payment card and decrypt the encrypted pass using the private key to retrieve the verified personal data of the adult consumer.

7. The system of claim 6, wherein the encrypted pass is generic to a plurality of aerosol-generating devices, and wherein the plurality of aerosol-generating devices includes the aerosol-generating device.

8. The system of claim 1, wherein the verified personal data of the adult consumer enables activation of at least one active ingredient in the aerosol-generating device.

9. The system of claim 8, wherein the at least one active ingredient includes at least one of nicotine, caffeine, and a cannabinoid.

10. The system of claim 1, wherein the aerosol-generating device includes an aerosol-forming substrate, a heater configured to heat the aerosol-forming substrate, and a power source configured to provide power to the heater.

11. The system of claim 1, wherein the aerosol-generating device includes a pre-vapor formulation, a heater configured to vaporize the pre-vapor formulation, and a power source configured to provide power to the heater.

12. The system of claim 1, wherein:
the second wireless communication chip of the aerosol-generating device includes a wireless reader; and
the first wireless communication chip of the physical document includes a wireless tag detectable by the wireless reader of the aerosol-generating device to initiate reception of the verified personal data.

13. A method for unlocking an aerosol-generating device, the method comprising:
receiving, from a wireless communication chip of a physical document issued and unique to an adult consumer, verified personal data of the adult consumer;
determining whether the adult consumer meets an age condition based on the verified personal data; and
unlocking the aerosol-generating device in response to the adult consumer meeting the age condition.

14. The method of claim 13, wherein the physical document is a government-issued identification.

15. The method of claim 14, wherein the government-issued identification is one of a biometric passport or a biometric driver's license.

16. The method of claim 13, wherein the physical document is a payment card.

17. The method of claim 16, wherein the payment card is a contactless bank card.

18. The method of claim 16, wherein:
the payment card includes memory configured to store an encrypted pass with the verified personal data of the adult consumer;
receiving verified personal data of the adult consumer includes receiving the encrypted pass from the payment card; and
determining whether the adult consumer meets an age condition based on the verified personal data includes decrypting the encrypted pass using a private key stored on the aerosol-generating device to retrieve the verified personal data of the adult consumer.

19. The method of claim 13, further comprising activating at least one active ingredient in the aerosol-generating device based on the verified personal data.

20. The method of claim 19, wherein the at least one active ingredient includes at least one of nicotine, caffeine, and a cannabinoid.

21. The method of claim 13, wherein:
the aerosol-generating device includes a wireless reader;
the wireless communication chip of the physical document includes a wireless tag; and
the method further comprises detecting, by the wireless reader, the wireless tag to initiate reception of the verified personal data.
